(19) United States
(12) Patent Application Publication
(10) Pub. No.: US 2026/0295255 A1
(43) Pub. Date: Oct. 1, 2026

(54) SMART MEDICAL DEVICE INSERTION

(71) Applicant: COCHLEAR LIMITED, Macquarie University, NSW (AU)

(72) Inventor: Peter GIBSON, Macquarie University (AU)

(21) Appl. No.: 19/477,942

(22) PCT Filed: Apr. 23, 2024
(86) PCT No.: PCT/IB2024/053961

§ 371 Date: Oct. 23, 2025

Related U.S. Application Data
(60) Provisional application No. 63/461,541, filed on Apr. 24, 2023.

(51) Int. Cl. A61N 1/36; A61B 34/10; A61B 34/30; A61B 34/32; A61N 1/05; G16H 40/63

(52) U.S. Cl. CPC A61N 1/36039; A61B 34/32; A61N 1/0541; G16H 40/63; A61B 2034/107; A61B 2034/302; A61N 1/0543; A61N 1/36046

(57) ABSTRACT

A method, including advancing, as part of an implantation procedure into a human, at least a first portion of an electrode array into a cochlea of the human during a first temporal period, providing information to a computer system, the provided information including spatial based data relating to the electrode array during the implantation procedure of the electrode array into the human, receiving information based on an evaluation by the computer system of the provided information, the evaluation having taken into account a history of the spatial based data provided in the provided information, the received information being a recommendation as to how to move the electrode array during second temporal period following the first temporal period and moving the electrode array according to the received information.

### CROSS-REFERENCE TO RELATED APPLICATIONS

This application claims priority to U.S. Provisional Application No. 63/461,541, entitled SMART MEDICAL DEVICE INSERTION, filed on Apr. 24, 2023, naming Peter GIBSON as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In accordance with an exemplary embodiment, there is a method, comprising:
advancing, as part of an implantation procedure into a human, at least a first portion of an electrode array into a cochlea of the human during a first temporal period;
providing information to a computer system, the provided information including spatial based data relating to the electrode array during the implantation procedure of the electrode array into the human;
receiving information based on an evaluation by the computer system of the provided information, the evaluation having taken into account a history of the spatial based data provided in the provided information, the received information being a recommendation as to how to move the electrode array during second temporal period following the first temporal period; and
moving the electrode array according to the received information.

In an embodiment, there is a non-transitory computer readable medium having recorded thereon, a computer program for executing at least a portion of a method, the computer program including:
code for receiving input based on at least one of past movement, past pose, past status, current movement, current pose or current status of an electrode array at least partially in a human during an implantation procedure of the electrode array in the human; and
code for automatically analyzing data based on at least a portion of the input to develop a recommend action to be taken in the implantation procedure, wherein the analyzed data includes:
   a current position of the electrode array; and
   at least one of:
      at least one movement of the array during a first temporal period prior to a current temporal period;
      at least one past pose of the array during the first temporal period; or
   at least one past status of the electrode array during the first temporal period.

In an embodiment, there is a system, comprising an input suite configured to receive input based on a spatial feature of an implantable medical device during implantation into a human; an output suite; and a data analysis computer in signal communication with the input suite and the output suite, the data analysis computer including circuitry and being configured to automatically analyze, at least in part with the circuitry, the received input as that input relates to a history of spatial features of the medical device during implantation and automatically develop, based on the analysis, a subsequent movement of the medical device to be implemented during the implantation into the human, wherein the system is configured to at least one of automatically output data based on the developed movement via the output suite or move the medical device based on the developed movement via the output suite.

In an embodiment, there is an apparatus, comprising: a cochlear implant electrode array insertion device, wherein the device includes: an input component configured to receive data based on data related to an insertion process of a cochlear implant electrode array, the received data being received in real time relative to the process; and at least one of:
an output component configured to provide output to a user regarding an action to take with respect to implanting the array in a human; or
an actuator configured to move the array relative to the human in
an automated manner, wherein
   the device at least one of (i) includes non-transitory logic or (ii) has access to non-transitory logic and/or results of analysis of non-transitory logic, wherein the non-transitory logic least one of identifies the action to take based on the received data, the received data being related to the insertion process including a prior action of the actuator if present, or another actuator configured to move the array in a manually controlled manner and/or a prior movement of the electrode array or controls the actuator based on the prior action of the actuator and/or a prior movement of the electrode array.

In an embodiment, there is a method, comprising:
obtaining (i) medical device positional data and (ii) medical device movement data for a statistically significant number of medical device implantation processes on a human for the medical device; and
analyzing the obtained data to develop a predictive algorithm for a current medical device movement behavior based on the results of the analysis, wherein the predictive algorithm predicts future movement of the current medical device based on input specific to the current medical device, the current medical device not being one of the medical devices of the medical device implantation processes.

Also, in an embodiment, there is a cochlear implant electrode array insertion robot, including:
a digital and/or analog input jack configured to receive digital and/or analog signals related to an insertion process of a cochlear implant electrode array, the received signals being received in real time relative to the process; and
   at least one of:
      an audio-visual device configured to provide output to a user regarding an action to take with respect to implanting the array in a human; or
      an actuator configured to move the array relative to the human in an automated manner, wherein
the device includes non-transitory logic and/or results of analysis of non-transitory logic, wherein the non-transitory logic least one of identifies the action to take based on the received data, the received data being related to the insertion process including a prior action of the actuator if present, or another actuator configured to move the array in a manually controlled manner and/or a prior movement of the electrode array or controls the actuator based on the prior action of the actuator and/or a prior movement of the electrode array.

### BRIEF DESCRIPTION OF THE DRAWINGS

Embodiments are described below with reference to the attached drawings, in which:

FIGS. 1A-1C are views of exemplary prosthetic devices;

FIG. 2 is a side view of an embodiment of an insertion guide for implanting a cochlear implant electrode assembly such as the electrode assembly illustrated in FIG. 1;

FIGS. 3A and 3B are side and perspective views of an electrode assembly extended out of an embodiment of an insertion sheath of the insertion guide illustrated in FIG. 2;

FIGS. 4A-4E are simplified side views depicting the position and orientation of a cochlear implant electrode assembly insertion guide tube relative to the cochlea at each of a series of successive moments during an exemplary implantation of the electrode assembly into the cochlea;

FIGS. 4F-4I show side views depicting position and orientation of a cochlear implant electrode array;

FIG. 5A is a side view of a perimodiolar electrode assembly partially extended out of a conventional insertion guide tube showing how the assembly may twist while in the guide tube;

FIG. 6A is a cross-sectional view of a conventional electrode assembly;

FIG. 6B is a cross-sectional view of the conventional electrode assembly of FIG. 6C positioned in the insertion guide tube;

FIGS. 7A to 9G show views relating to electrode array position and orientation;

FIGS. 10 to 13 and 15 and 16 show exemplary flowcharts;

FIG. 14 shows a schematic of a device; and

FIGS. 17-38 present some exemplary embodiments of hardware for implementing some of the teachings detailed herein.

### DETAILED DESCRIPTION

Merely for ease of description, the techniques presented herein are sometimes described herein with reference to an illustrative medical device, namely a cochlear stimulator, and in other instances, a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. Some embodiments include the utilization of the teachings herein to treat an inner ear of a recipient that has and/or utilizes one or more of these devices. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. In further embodiments, the techniques presented herein may be used with air purifiers or air sensors (e.g., automatically adjust depending on environment), hospital beds, identification (ID) badges/bands, or other hospital equipment or instruments.

The teachings detailed herein can be implemented in sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with all of or parts of a cochlear implant, cochlear stimulator, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

Thus, merely for ease of description, the first illustrative medical device is a hearing prosthesis. Any techniques presented herein described for one type of hearing prosthesis or any other device disclosed herein corresponds to a disclosure of another embodiment of using such teaching with another device (and/or another type of hearing device including other types of bone conduction devices (active transcutaneous and/or passive transcutaneous), middle ear auditory prostheses (particularly, the EM vibrator/actuator thereof), direct acoustic stimulators), etc. The techniques presented herein can be used with implantable/implanted microphones (where such is a transducer that receives vibrations and outputs an electrical signal (effectively, the reverse of an EM actuator), whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), and thus any disclosure herein is a disclosure of utilizing such devices with the teachings herein (and vice versa), providing that the art enables such. The teachings herein can also be used with conventional hearing devices, such as telephones and ear bud devices connected MP3 players or smart phones or other types of devices that can provide audio signal output, that use an EM transducer. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

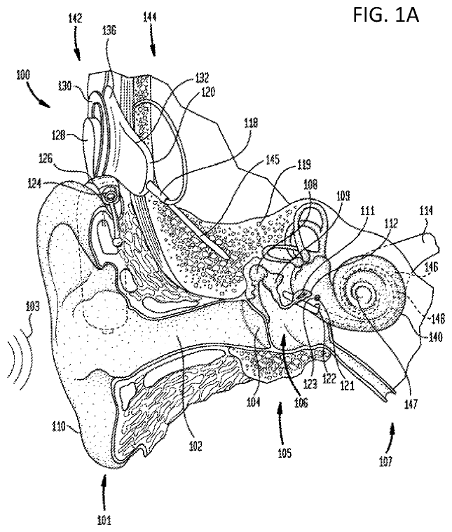

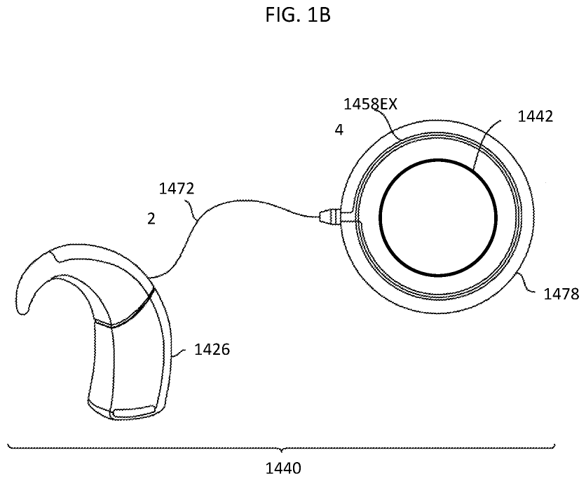

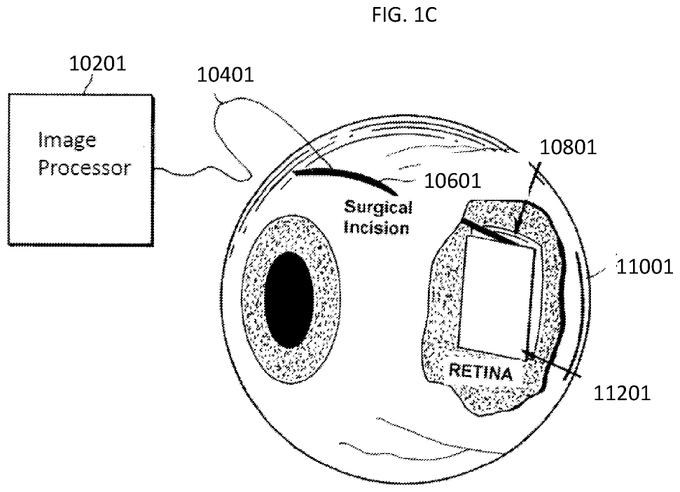

FIG. 1A is a perspective view of an exemplary cochlear implant 100 implanted in a recipient having an outer ear 101, a middle ear 105, and an inner ear 107. In a fully functional ear, outer ear 101 comprises an auricle 110 and an ear canal 102. Acoustic pressure or sound waves 103 are collected by auricle 110 and channeled into and through ear canal 102. Disposed across the distal end of ear canal 102 is a tympanic membrane 104 that vibrates in response to sound waves 103. This vibration is coupled to oval window or fenestra ovalis 112 through the three bones of the middle ear 105, collectively referred to as the ossicles 106, and comprising the malleus 108, the incus 109, and the stapes 111. Ossicles 106 filter and amplify the vibrations delivered by tympanic membrane 104, causing oval window 112 to articulate, or vibrate. This vibration sets up waves of fluid motion of the perilymph within cochlea 140. Such fluid motion, in turn, activates hair cells (not shown) inside the cochlea which in turn causes nerve impulses to be generated which are transferred through spiral ganglion cells (not shown) and auditory nerve 114 to the brain (also not shown) where they are perceived as sound.

The exemplary cochlear implant illustrated in FIG. 1A is a partially implanted stimulating medical device. Specifically, cochlear implant 100 comprises external components 142 attached to the body of the recipient, and internal or implantable components 144 implanted in the recipient. External components 142 typically comprise one or more sound input elements for detecting sound, such as microphone 124, a sound processor (not shown), and a power source (not shown). Collectively, these components are housed in a behind-the-ear (BTE) device 126 in the example depicted in FIG. 1A. External components 142 also include a transmitter unit 128 comprising an external coil 130 of a transcutaneous energy transfer (TET) system. Sound processor 126 processes the output of microphone 124 and generates encoded stimulation data signals which are provided to external coil 130.

Internal components 144 comprise an internal receiver unit 132 including a coil 136 of the TET system, a stimulator unit 120, and an elongate stimulating lead assembly 118. Internal receiver unit 132 and stimulator unit 120 are hermetically sealed within a biocompatible housing commonly referred to as a stimulator/receiver unit. Internal coil 136 of receiver unit 132 receives power and stimulation data from external coil 130. Stimulating lead assembly 118 has a proximal end connected to stimulator unit 120, and extends through mastoid bone 119. Lead assembly 118 has a distal region, referred to as electrode assembly 145, a portion of which is implanted in cochlea 140.

Electrode assembly 145 can be inserted into cochlea 140 via a cochleostomy 122, or through round window 121, oval window 112, promontory 123, or an opening in an apical turn 147 of cochlea 140. Integrated in electrode assembly 145 is an array 146 of longitudinally-aligned and distally extending electrode contacts 148 for stimulating the cochlea by delivering electrical, optical, or some other form of energy. Stimulator unit 120 generates stimulation signals each of which is delivered by a specific electrode contact 148 to cochlea 140, thereby stimulating auditory nerve 114.

FIG. 1B depicts an exemplary external component 1440. External component 1440 can correspond to external component 142 of the system 10 (it can also represent other body worn devices herein/devices that are used with implanted portions). As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

FIG. 1C presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of an array for a hearing prosthesis corresponds to a disclosure of an array for a retinal prosthesis. Any disclosure herein of a method of using or operating or otherwise working with a hearing prosthesis herein corresponds to a disclosure of using or operating or otherwise working with a retinal prosthesis in an analogous manner.

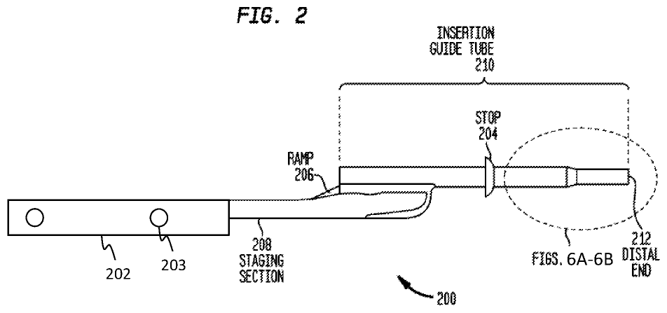

Electrode assembly 145 may be inserted into cochlea 140 with the use of an insertion guide. FIG. 2 is a side view of an embodiment of an insertion guide for implanting an elongate electrode assembly generally represented by electrode assembly 145 into a mammalian cochlea, represented by cochlea 140. The illustrative insertion guide, referred to herein as insertion guide 200, includes an elongate insertion guide tube 210 configured to be inserted into cochlea 140 and having a distal end 212 from which an electrode assembly is deployed. Insertion guide tube 210 has a radially-extending stop 204 that may be utilized to determine or otherwise control the depth to which insertion guide tube 210 is inserted into cochlea 140.

Insertion guide tube 210 is mounted on a distal region of an elongate staging section 208 on which the electrode assembly is positioned prior to implantation. A robotic arm adapter 202 is mounted to a proximal end of staging section 208 to facilitate attachment of the guide to a robot, which adapter includes through holes 203 through which bolts can be passed so as to bolt the guide 200 to a robotic arm, as will be detailed below. During use, electrode assembly 145 is advanced from staging section 208 to insertion guide tube 210 via ramp 206. After insertion guide tube 210 is inserted to the appropriate depth in cochlea 140, electrode assembly 145 is advanced through the guide tube to exit distal end 212 as described further below.

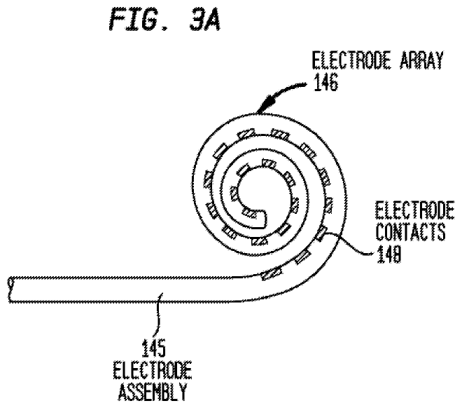

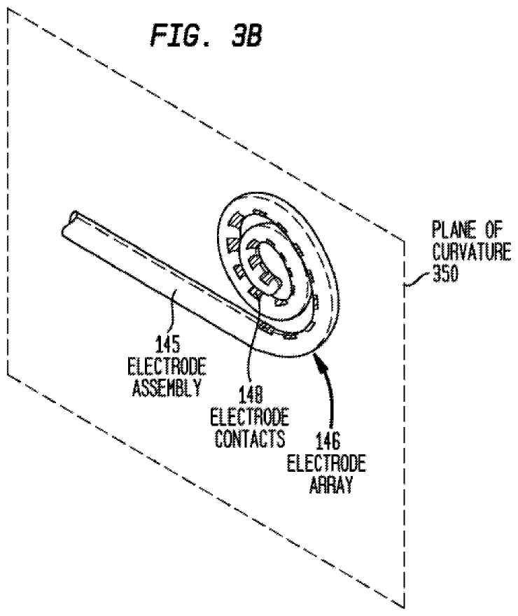

FIGS. 3A and 3B are side and perspective views, respectively, of representative electrode assembly 145. As noted, electrode assembly 145 comprises an electrode array 146 of electrode contacts 148. Electrode assembly 145 is configured to place electrode contacts 148 in close proximity to the ganglion cells in the modiolus. Such an electrode assembly, commonly referred to as a perimodiolar electrode assembly, is manufactured in a curved configuration as depicted in FIGS. 3A and 3B. When free of the restraint of a stylet or insertion guide tube, electrode assembly 145 takes on a curved configuration due to it being manufactured with a bias to curve, so that it is able to conform to the curved interior of cochlea 140. As shown in FIG. 3B, when not in cochlea 140, electrode assembly 145 generally resides in a plane 350 as it returns to its curved configuration. That said, it is noted that embodiments of the insertion guides detailed herein and/or variations thereof can be applicable to a so-called straight electrode array, which electrode array does not curl after being free of a stylet or insertion guide tube etc., but instead remains straight.

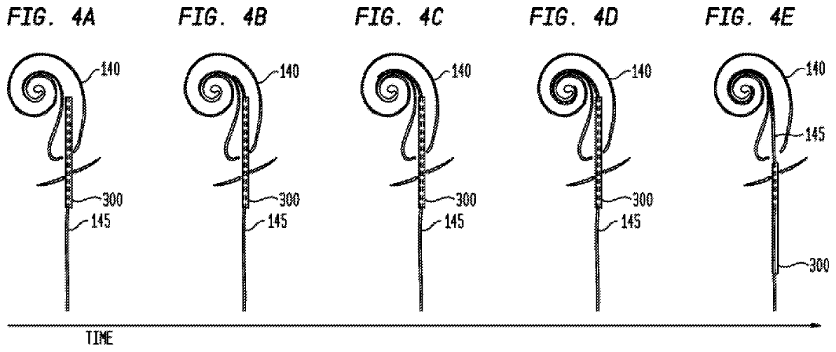

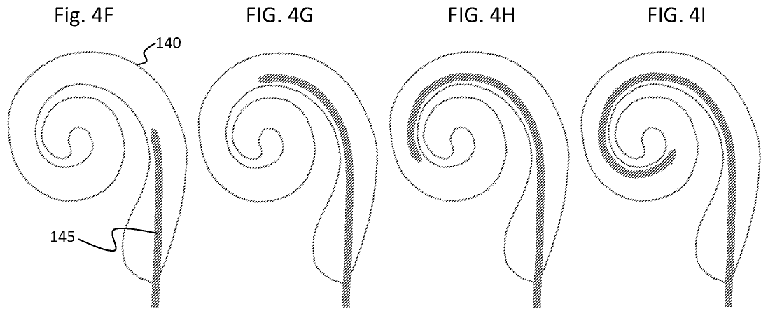

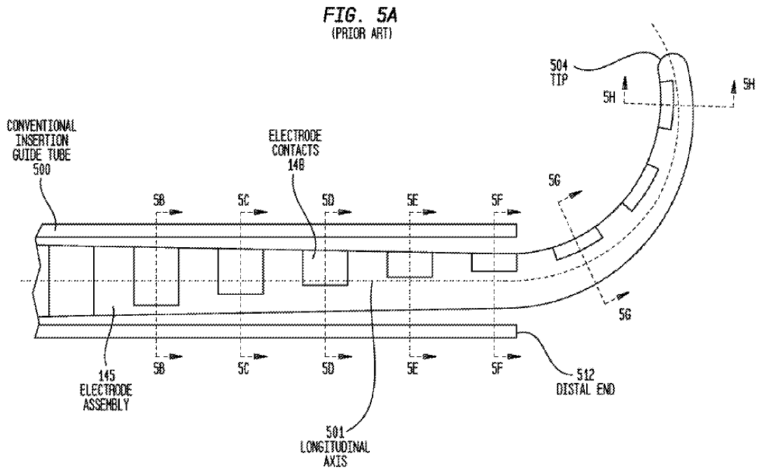

FIGS. 4A-4E are a series of side-views showing consecutive exemplary events that occur in an exemplary implantation of electrode assembly 145 into cochlea 140. Initially, electrode assembly 145 and insertion guide tube 310 are assembled. For example, electrode assembly 145 is inserted (slidingly or otherwise) into a lumen of insertion guide tube 300. The combined arrangement is then inserted to a predetermined depth into cochlea 140, as illustrated in FIG. 4A. Typically, such an introduction to cochlea 140 is achieved via cochleostomy 122 (FIG. 1) or through round window 121 or oval window 112. In the exemplary implantation shown in FIG. 4A, the combined arrangement of electrode assembly 145 and insertion guide tube 300 is inserted to approximately the first turn of cochlea 140.

As shown in FIG. 4A, the combined arrangement of insertion guide tube 300 and electrode assembly 145 is substantially straight. This is due in part to the rigidity of insertion guide tube 300 relative to the bias force applied to the interior wall of the guide tube by pre-curved electrode assembly 145. This prevents insertion guide tube 300 from bending or curving in response to forces applied by electrode assembly 145, thus enabling the electrode assembly to be held straight, as will be detailed below.

As noted, electrode assembly 145 is biased to curl and will do so in the absence of forces applied thereto to maintain the straightness. That is, electrode assembly 145 has a memory that causes it to adopt a curved configuration in the absence of external forces. As a result, when electrode assembly 145 is retained in a straight orientation in guide tube 300, the guide tube prevents the electrode assembly from returning to its pre-curved configuration. This induces stress in electrode assembly 145. Pre-curved electrode assembly 145 will tend to twist in insertion guide tube 300 to reduce the induced stress. In the embodiment configured to be implanted in scala tympani of the cochlea, electrode assembly 145 is pre-curved to have a radius of curvature that approximates the curvature of medial side of the scala tympani of the cochlea. Such embodiments of the electrode assembly are referred to as a perimodiolar electrode assembly, and this position within cochlea 140 is commonly referred to as the perimodiolar position. In some embodiments, placing electrode contacts in the perimodiolar position provides utility with respect to the specificity of electrical stimulation, and can reduce the requisite current levels thereby reducing power consumption.

As shown in FIGS. 4B-4D, electrode assembly 145 may be continually advanced through insertion guide tube 300 while the insertion sheath is maintained in a substantially stationary position. This causes the distal end of electrode assembly 145 to extend from the distal end of insertion guide tube 300. As it does so, the illustrative embodiment of electrode assembly 145 bends or curves to attain a perimodiolar position, as shown in FIGS. 4B-4D, owing to its bias (memory) to curve. Once electrode assembly 145 is located at the desired depth in the scala tympani, insertion guide tube 300 is removed from cochlea 140 while electrode assembly 145 is maintained in a stationary position. This is illustrated in FIG. 4E.

In an embodiment, the goal of the insertion process is to achieve the insertion regime seen in FIGS. 4A-4E, at least for a pre-curved array.

And note that embodiments include insertion of electrode arrays without a guide tube or a guide device whatsoever. FIGS. 4F to 4I show such an insertion. These figures present a series of diagrams that show an optimal insertion of a pre-curved perimodiolar electrode array at different points during insertion. In this example the electrode takes an optimal path around the modiolus, maintaining proximity to the modiolus throughout the insertion.

Returning to FIGS. 3A-3B, perimodiolar electrode assembly 145 is pre-curved in a direction that results in electrode contacts 148 being located on the interior of the curved assembly, as this causes the electrode contacts to face the modiolus when the electrode assembly is implanted in or adjacent to cochlea 140. Insertion guide tube 500 retains electrode assembly 145 in a substantially straight configuration, thereby preventing the assembly from taking on the configuration shown in FIG. 3B.

FIG. 5A is a side view of perimodiolar electrode assembly 145 partially extended out of a conventional insertion guide tube 500, showing how the assembly may twist while in the guide tube.

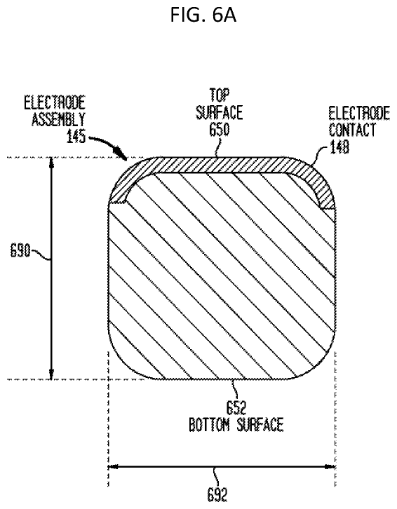

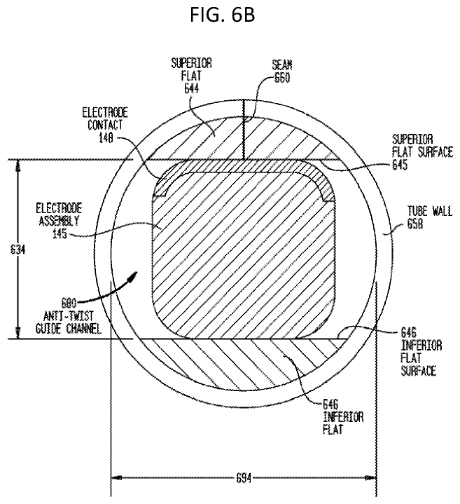

As shown in FIG. 6A, electrode assembly 145 has a rectangular cross-sectional shape, with the surface formed in part by the surface of the electrode contact, referred to herein as top surface 650, and the opposing surface, referred to herein as bottom surface 652, are substantially planar. In other embodiments, the array is circular or ovaloid in cross-section.

FIG. 6B shows a cross-section of the insertion guide tube and the electrode assembly 145. Tube wall 658 has surfaces 644 and 646 which extend radially inward to form an anti-twist guide channel 680. Specifically, a superior flat 644 provides a substantially planar lumen surface along the length of a section of the tube. Superior flat 644 has a surface that is substantially planar and which therefore conforms with the substantially planar top surface 650 of electrode assembly 145. Similarly, inferior flat 646 has a surface that is substantially planar which conforms with the substantially planar bottom surface 652 of electrode assembly 145. As shown in FIG. 6D, when a distal region of electrode assembly 145 is located in anti-twist section 620, the surfaces of superior flat 644 and inferior flat 646 are in physical contact with top surface 650 and bottom surface 652, respectively, of the electrode assembly. This prevents the electrode assembly from curving.

With the above as context, embodiments include cochlear implant electrode array implantation techniques that include providing input or otherwise guidance/recommendations to a surgeon or other professional who is inserting the array into the cochlea, in real time, while the healthcare professional is inserting the array into the cochlea.

It is briefly noted that embodiments are not limited to insertion of an electrode array into a cochlea. Any disclosure herein related to an electrode array corresponds to another alternate disclosure of an insertion of another device, into a cochlea, for the purposes of textual economy, providing that such is utilitarian value providing that the art enables such. Corollary to this is that any disclosure herein related to a cochlea corresponds to an alternate disclosure of another cavity within a human, provided that the art enables such and providing that there is utilitarian value with respect to applications of the teachings detailed herein there to.

Pre-curved perimodiolar electrode arrays are intended to follow a trajectory around the modiolus, staying close to the modiolar wall and away from the delicate structures at the lateral wall of the otic capsule. FIGS. 4A-4I show insertion of the array following such a trajectory.

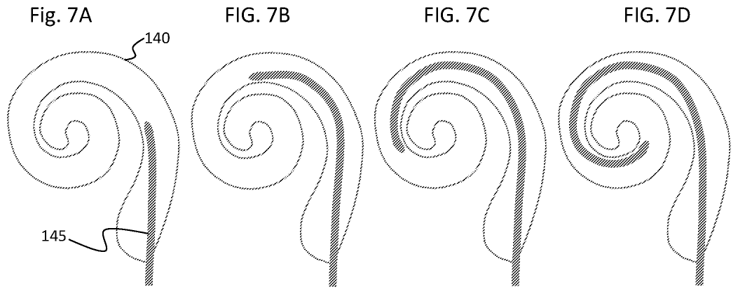

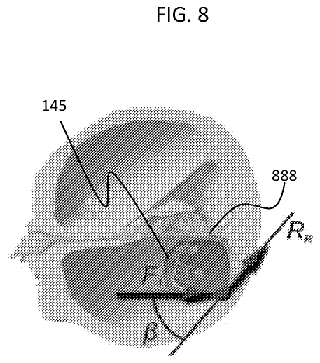

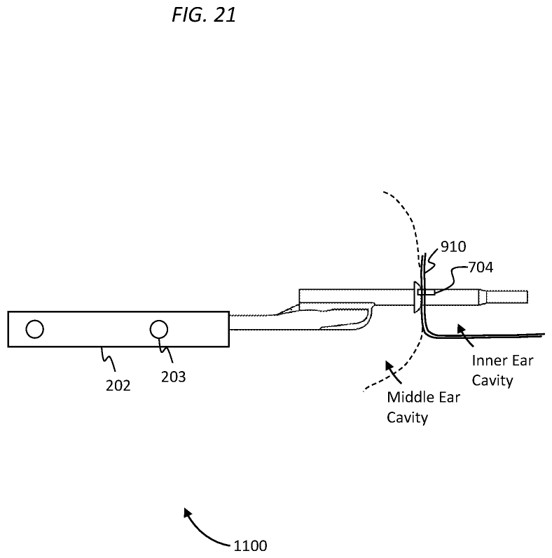

Typically, this is done blindly vis-à-vis what is actually happening in the cochlea with respect to the array, literally and often figuratively. In at least some exemplary embodiments there is no imaging that occurs during insertion with respect to what is going on in the cochlea. Corollary to this is that tactile response during the insertion process is open to too much judgment and otherwise can be misleading. Indeed, in the case of a pre-curved electrode array being inserted with the use of an insertion tube or insertion sheath, the friction of the array in the sheath dominates what the surgeon feels, leading to, in some instances, tactile blindness. Various features, including natural features, associated with the cochlea, such as for example a scalloped wall of the cochlea duct into which the array is being inserted, the angle of insertion, the friction associated with the surgical opening into the cochlea from the middle ear or from outside the cochlea and other natural features of the cochlea that can vary from human to human and can result in the tactile response being non-utilitarian with respect to understanding what is exactly happening within the cochlea with respect to progression of the electrode array therein. Corollary to this is that even when tactile response occurs, and if that tactile response was perfectly translatable and/or understandable by the surgeon, or even with imaging or some artificial method of determining the placement and/or prose of the electrode array in the cochlea in real time, the surgeon still may not have a lot of guidance or any guidance for that matter as to what should happen next with respect to insertion action relative to the next insertion action. In this regard, should the surgeon or other healthcare professional continue to insert the electrode array further, or should the surgeon stop and potentially retract the array? Should the surgeon twist the array, etc.? No guidance is given. It is totally up to the subjective and personally experience-based understanding of the surgeon as to what should be done next. And in at least some exemplary scenarios of insertion, sometimes pre-curved electrode arrays follow a less-optimal path. This is seen in FIGS. 7A-7D. In this scenario, the mid section of the array does not follow the modiolus during insertion. Friction at the tip prevents forward movement of the tip which causes the mid-section of the array to flex outward toward the delicate structures of the lateral wall. If the mid-section of the array presses on the lateral wall there is potential to cause disturbance or trauma to the delicate structures on the lateral wall. FIG. 7D shows the final position of the electrode array, where the array is not as close to the modiolus as in FIG. 4I (FIG. 4I showing the more utilitarian placement of the array). The scenario shown in FIGS. 7A-7D can occur due to a higher than normal level of friction at the tip, which can prevent the tip from moving forward the same distance as the insertion movement. The friction can be due to an uneven surface along the modiolar wall by way of example and not by way of limitation. Histology shows that some modular walls exhibit a scalloped surface which can catch the tip. When this happens the mid-section of the electrode array flexes out until there is sufficient forward push from the tensioned array to overcome the friction of the tip on the modiolus. In some cases, the mid-section of the electrode array can move out far enough to press on the delicate structures of the lateral wall and have potential to cause some disturbance or trauma to those structures. This can be not good in some instances. If an array presses on the lateral wall, there can be forces upward toward the basilar membrane 888, as seen in FIG. 8, where F₁ is the force that results from the electrode array boing outward away from the modiolus wall, and Rᵣ is the reaction force owing to the curvature of the modiolus wall that drives the electrode array upwards towards the basilar membrane 888, with β being the angle relative to F₁ and the resulting force Rᵣ. (FIG. 8 comes from It is from a paper by Prof. J Thomas Roland of NYU. A Model for Cochlear Implant Electrode Insertion and Force Evaluation: Results with a New Electrode Design and Insertion Technique, Laryngoscope 115: August 2005. FIG. 21.)

Even when a surgeon or otherwise a healthcare professional has reason to believe or suspect or otherwise has knowledge that there is an existing placement and/or pose an electrode array that can be deleterious at the moment or could result in a deleterious scenario in the future, as mentioned above, the surgeon or otherwise the healthcare professional (hereinafter, reference will be made to the surgeon, but such corresponds to an alternate disclosure in the interest of textual economy to another type of healthcare professional) has no guidance as to what action to next take. That is, for example, the surgeon does not have any guidance regarding what to do should an electrode tip stop or slow progression such that the mid-section of the array starts to flex out toward the lateral wall. Should the surgeon keep pushing forward in the hope that the tip will start to move? Should the surgeon pull back to retract the tip from the point of high friction and then re-start to insert? Embodiments can provide utilitarian value with respect to providing guidance to the surgeon on a next maneuver (e.g., the likely “best” next maneuver, based on statistics—more on this below) to take in the circumstances that exist at the moment.

Embodiments include insertion actions where the electrode array is inserted in a slow and/or step-wise fashion with some forward and, in some instances, some backward movements, where the movements (forward and/or backward) might be intentional or unintentional. An unintentional movement could happen for example if an electrode array is released and the stored elastic energy in the electrode lead (the part outside the cochlea) causes a movement.

When inserting or withdrawing the electrode array in some embodiments, with a number of forward and backward movements, in some embodiments, the array does not always follow the same path. There is a type of hysteresis effect that is present that can influence future movement. The next movement of an electrode array will not simply follow the contour that the electrode array traversed in the most recent movement, in some embodiments. The array might not continue to follow the contour that the array happens to be in at any point in time. The contour may be different to a previous contour based on the history of movement. For example, whether the most recent movements were to push forward or pull back or twist or a combination in some sequence. In an embodiment, there are a number of ways to withdraw the electrode array. For example, if the electrode array is still in the sheath, then one can pull the electrode array back through the sheath, or one can pull the sheath and electrode out together. These might have different effects depending on how far the electrode is inserted. Of course, if a sheath is not being used, one can pull the array by itself. Also, if the sheath has already been removed, then one can pull the electrode back with no sheath involved. Accordingly, in some embodiments, the recommendations/instructions can include any one or more of the aforementioned withdrawal techniques. Moreover, based on the data collected, the system could advise/recommend to remove the electrode array completely, reload into the sheath (if a sheath is being used), and start afresh. Accordingly, embodiments herein can include recommending the relationships of movements relative to the insertion sheath and/or can include completely starting over, whether such is with the use of the sheath or not. Indeed, it could be that a determination is made that the insertion sheath is not useful with respect to a particular insertion, and thus the system could recommend to not use the sheath/stop using the sheath, or alternatively, could recommend to use and insertion sheath if the procedure up until that point has been utilized without one

The contour an electrode array will take will also depend, in some embodiments, on the current location of the array in the cochlea. For example, if the array is resting on the modiolus, or flexed away from the modiolus, or at a point in the insertion where the tip has previously hung up on the modiolus, the next movement can result in a different contour. (Note that in various scenarios, the next movement may result in a fully inserted or partially inserted array.)

Embodiments include an automated arrangement that provides an indication that informs the surgeon on how to make and/or provides additional information to make a decision the next movement of the electrode array. This can improve a specific surgery, and can be based on an evaluation of the history of movement of the electrode array during the surgery and/or knowledge of the current position of an electrode array. The history of movement and/or the current position can be recorded in a computer system, such as a smart system, for analysis and development of a recommendation to the surgeon on the next step for insertion of the electrode array. And as will be described below, embodiments can include communicating with the implantable component and/or the external components of the hearing prostheses for example, and in an embodiment, data could be stored in a smart implant and the implant itself could contribute to the analysis. The smart implant can have a direct Bluetooth communication with an external device which could be a standard smart phone or tablet, by way of example, or any of the components herein. In an embodiment, it is the implant, or the external component of the prostheses or both, that performs the analysis or otherwise makes the recommendation, or at least performs part of the analysis. Any disclosure herein of a system that performs the analysis or otherwise evaluate the data or otherwise collects data or otherwise records the data or otherwise stores the data corresponds to a disclosure in the interest of textual economy of the implant and/or the external component doing so providing that the art enables such unless otherwise noted.

By way of exemplary scenario, the electrode array is practically fully inserted, or otherwise is more than a certain amount inserted (e.g., at least 60, 65, 70, 75, 80, 85, 90, or 95% of the length of the array that will ultimately be inserted at completion of the insertion process is inserted, or any value or range of values therebetween in 1% increments) and the most recent action was to insert further, and there is evidence that the mid-section of the array has moved away from the modiolus (which indication could be by way of imaging (e.g., CT scan), or electrical energizement of electrodes, or by the techniques of PCT Patent Application Publication No. WO 2021/028824, or WO 2018/173010, or WO 2019/162837 or WO 2019/175764 or US Patent Application Publication No. 2022/0016416 or US Patent Application Publication No. 2018/0140829 or US Patent Application Publication No. 2018/0050196, to inventor Nicolas Pawsey, Published on Feb. 22, 2018—in an embodiment, the teachings of those publications can be used to determine the spatial features and/or movement features detailed herein and/or any spatial feature and/or movement feature that can be determined using the teachings of those references can be used herein for the spatial features and/or movement features). In an embodiment, the electrodes of the electrode array can be energized and read electrodes thereof can be used to sense spatial features/movement features, and this sensed data can be provided to the systems disclosed herein—more on this below.

An indication can be provided to the surgeon that the tip of the electrode is caught and/or an indication can be provided to the surgeon that the next action should be (or at least carefully contemplated to be) to pull back. If this takes place, the system could measure or otherwise attempt to evaluate if this brings the mid-section closer. This could be repeated (insert an action and withdraw an action) to confirm that this has brought the mid-section closer. The system could provide an instruction to (or provide a recommendation to—any disclosure of an instruction corresponds to a disclosure of a recommendation, and vice versa, unless otherwise noted) the surgeon to repeat the action just noted, so the system can check for the results. An input system could permit the surgeon to notify the system that he/she is repeating (or the system could use the sensors thereof to infer/deduce that such is the case). Then, for example, if it is not possible to insert the tip further, the system could provide guidance instructing the surgeon to retract the electrode array to a location (one that is deemed to result in the most utilitarian position of the electrode array relative to the modiolus wall for example) which can be specified by the system or otherwise embodied in instructions on how to move the array (the surgeon may not know what is happening per se, only that he/she should do such) and then leave the electrode array in that place to optimize proximity of the electrode to the modiolus. And note that embodiments can include breaking up the insertion into phases. A first phase could cover the introduction of the sheath to an optimal depth and angle to facilitate advancing the electrode out of the sheath with the optimal vector to avoid going too wide of the modiolus. The second phase could cover the advancement of the electrode array until it has reached the full depth of insertion and ensuring during this phase that the electrode array does not bow outward and cause trauma to the lateral wall on the way, and embodiments herein can be directed to avoiding such (damage to the lateral wall), and reversing the bowing/eliminating such if such occurs. And in the third phase, the array is at or around full insertion, and this phase can including optimizing/perfecting as best possible the final position of the electrode array as close as possible to the modiolus. During phase 2, a goal can be to avoid the electrode array impinging on the lateral wall, or at least limiting the amount of force thereon. In some embodiments, it is not essential to be as close as possible to the modiolus, however being far away from the lateral wall (and hence close as possible to the modiolus) gives more margin for error and room for adjustment of the insertion movements. During phase 3, a goal can be to have the array end up as close as possible to the modiolus, and while attempting to execute this, avoid pushing out onto the lateral wall. Embodiments, can thus include recommending movements and actions etc., including the application of force or the reduction of force onto the array in the direction of insertion and/or in the direction of withdrawal, so as to avoid the array impinging on the lateral wall and/or to reduce the likelihood of such and/or at least reduce the amount of force applied to the lateral wall relative to that which would otherwise be the case. Embodiments thus can include devices systems and methods that can forecast or otherwise estimate what actions would result in the array moving towards the lateral wall or contacting the lateral wall or increase the force onto the lateral wall or otherwise results in a force that is unacceptably high, and thus recommend actions to take that would reduce the likelihood or otherwise avoid such. Embodiments can also include devices systems and methods that can recommend what actions to take to keep the array is closed as possible to the modiolus and/or avoid pushing the array out onto the lateral wall. Embodiments can thus include devices systems and methods that can forecast what actions would result in the array moving away from the modiolus or otherwise pushing the array outward onto the lateral wall and thus recommend actions to take that would reduce the likelihood or otherwise avoid such and what actions to take that will result in the array being as close as possible to the modiolus wall.

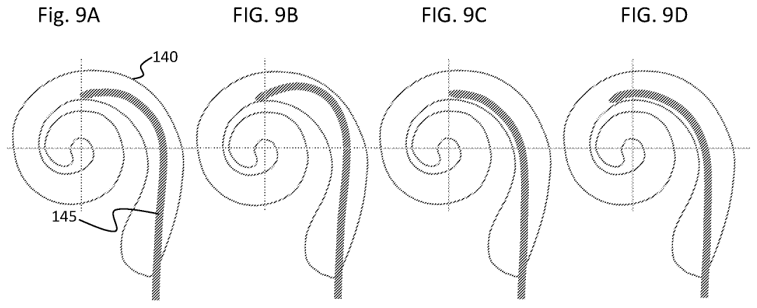

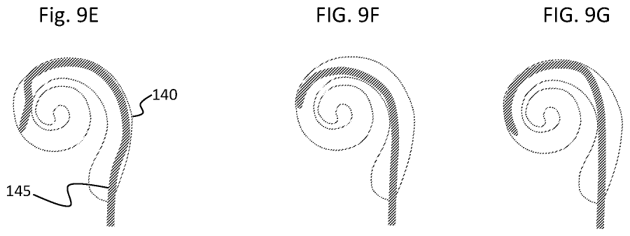

In an exemplary scenario, if a modiolar electrode array has been inserted to a point where the tip is slowed or stopped, the system can be configured to provide information or otherwise note the point in the cochlea where this has happened. Because the system can detect/determine the pose of the array, the system can deduce/determine that the pose of the electrode array indicates that the tip is caught. The system can then provide guidance to the surgeon to retract the electrode to free up the tip and then restart insertion. In the retraction step, the system can confirm that the tip has pulled back away from the point it was caught. Then in the re-insertion, the system can monitor the movement and location of the tip to confirm if it has gone past the point where the tip became slowed or caught. The system can then advise/recommend to the surgeon to continue to insert the electrode. FIGS. 9A-9D show, schematically, an example of this scenario, where the tip initially catches (FIGS. 9A and 9B) and then the array 145 is retracted so that the tip is pulled back from the point at which it was caught as seen in FIG. 9C. The pose/contour of the array in FIG. 9C is different to that in FIG. 9A so that when the electrode array is advanced, the tip is able to go past the point where the tip became caught. Embodiments include a system that can measure or otherwise determine the pose/contour as the electrode array is pulled back and noting/determining that the pose/contour is different from that which was the case before (FIG. 9A vs. FIG. 9C), and embodiments can include a system that is configured to, based on that measurement, advise the surgeon to re-start insertion of the electrode array which is then able to go past the point where the tip originally became caught (FIG. 9D.) And briefly, while it is noted that the teachings herein are often described relating to a perimodiolar electrode array, the teachings are also applicable to a straight electrode array in an alternate embodiment. FIGS. 9E-9G show an exemplary scenario as applied to a straight electrode array. These figures depict an example of a straight array insertion using the concepts described herein. FIG. 9E shows the tip lodging in the lateral wall and buckle begins with arching toward the medial wall. On determining that the tip has stopped, the system can note the point where the electrode tip lodged and provide guidance to retract the electrode. FIG. 9F shows the electrode upon a small retraction (which can be instructed by the system). The system can note that the tip has pulled back from the point at which it became caught. Note that for a straight electrode array, the array will tend to pull toward the modiolar wall in the mid-section of the array when it is retracted. This is a change in pose/contour of the array compared to that during insertion. When the system detects that the buckling has been resolved, and the tip has moved back from the point where it was caught, the system can provide guidance to re-commence insertion of the electrode. The system can then continue to monitor the pose/contour of the array as it is inserted to confirm the progress of the insertion. FIG. 9G shows the electrode traversing past the original point where the electrode tip was caught.

The system can also monitor the pose/contour of the electrode during the insertion and note and/or indicate the pose/contour at the point that the tip of the electrode becomes slowed or caught. Then in the case of a retraction and re-insertion, the system could compare whether the pose is the same or different to that which was the case when the tip first became caught. When the pose changes with retraction, the system can give guidance to re-start insertion of the array. If the pose remains different as the tip reaches the point where the tip first became caught, the system could provide guidance to continue to insert, or otherwise an indication that the prior “caught” scenario has not been repeated and/or that the tip has advanced further than before. If on re-insertion, the pose/contour is the same as the original when the tip became caught, the system could recommend a different maneuver on a second or third attempt to insert the electrode.

In view of the above, embodiments can address scenarios where, when inserting or withdrawing electrode arrays (partially or fully), the array does not always follow the same path and/or where there can be a hysteresis effect and/or the movement of the electrode array can depend on the status of the array (e.g., fully inserted, partially inserted). Embodiments include devices, systems, and/or methods that measure and/or interpret modiolar proximity, and enable adjustment of the array, where a current insertion depth, the recent history of movement of the array, and/or what type of adjustment was being made to the electrode array in the last movement are known, and taken into account to provide guidance/instruction on the next movement.

Embodiments can break down, for evaluation purposes, in real time, literally or computationally (e.g., akin to a finite element analysis of a circle or a curved surface (a series of facets—in some embodiments, so minute that for all practical purposes, the facets are indistinguishable from a smooth surface)), the movement of the electrode array in relatively discrete and small steps. Embodiments can then use this breakdown to analyze the past and the present status of the array, and automatically deduce a recommended next action. By way of example, if the electrode array is practically fully inserted and/or inserted by any of the amounts noted above for example, and the most recent action was to insert the array further and there is reason to believe (evidence, or otherwise, such as because the algorithm used to analyze the status/pose indicates such, or indicates a possibility of such) that the mid-section of the array has moved away from the modiolus (in absolute terms, or by an amount that is deemed undesirable or otherwise problematic, or outside a predetermined tolerance), then the system/algorithm would potentially indicate, for example, that the next action should be to pull back the array and measure or evaluate if this brings the mid-section of the array closer. The system/algorithm could recommend that this could be repeated (insert a step and withdraw a step) to confirm and then leave the electrode in that place. The system could then make a determination that if, however, the electrode array is only half inserted, and there is an indication that the apical electrodes are moving away from the modiolus (based on the analysis), this could be due to the tip becoming caught on the modiolus (the analysis could automatically determine this based on inputted data). In this instance, for example, the system/algorithm could recommend that pulling back on the array should bring the apical electrodes and/or the mid electrodes closer to the modiolus. (Note also that for a pre-curved perimodiolar electrode array, it is possible for the tip to be stubbed into the modiolus and the most proximal section to the tip to be away from the modiolus.) In an embodiment, it is the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th} or 15^{th} or any one or more of those electrodes, where the most apical electrode is the first, that is moved towards the modiolus. However, in this exemplary scenario, the electrode array is only partly inserted, so the goal is to release the tip from being caught and then insert further, and the system/algorithm would determine that based on the data. In this instance, the system/algorithm could determine that it is utilitarian to withdraw the electrode array by a significant distance until there is evidence that the tip is starting to pull back out, thus indicating (based on the algorithm/analysis) that the tip is released from whatever has caught the tip. The system could then analyze the data (where, in some embodiments, the data is constantly updated or otherwise updated at least every 5, 4, 3.5, 3, 2.5, 2, 1.5, 1, 0.9, 0.8, 0.7, 0.6, 0.5, 0.4, 0.3, 0.2, 0.1, 0.075, 0.05, 0.025, 0.02, 0.01 seconds or less, or any value or range of values therebetween in 1 millisecond increments), and after this, based thereon, indicate that the electrode array should be pushed further to get the tip past the stoppage. In an embodiment, the system could evaluate the location of the array relative to the modiolus upon the further pushing, and ratification of this instruction could be indicated by the electrode array going deeper without pushing out from the modiolus (at least within the tolerances). Conversely, if the array pushes out, the system could warn the user to stop and provide another recommendation as to what to do. And embodiments include ascertaining the pose of the electrode array at a given time/point during insertion, and obtaining data based on the resting state of the array in free air, and based thereon, there are devices and systems that calculate the elastic forces at that time (because the contact points with the inner ear and the sheath keep the electrode in a different position to the resting state) and utilize a computational model that can estimate the dynamics of movement as the electrode array is inserted or withdrawn from that point. Accordingly, embodiments include the rest-state design of the electrode array/features associated therewith, such as a model of the array (e.g., a spring model), or otherwise data based thereon as one of the inputs into the system and the difference between the current pose and the rest state can be one of the analytical actions. That is, by way of example, the devices and systems and methods herein can be utilized to predict the future movements and/or positions or otherwise future spatial data associated with the electrode array based on the past and/or current positions and/or movements and based on the known properties of the array or otherwise the model of the array. Thus, an embodiment includes calculating the elastic forces at a given point in time and therefore the dynamics of movement as the electrode array is moved in or out. Knowing this, the rest-state design of the electrode array can be included as one of the inputs of the inputs and the difference between the current pose and the rest state can be one of the analytical actions. And also note that in some scenarios, it is possible that the nature of the system of a quite weak spring (electrode array) and elastic tissue may lend itself to sometimes simply waiting without movement in or out, as the two weak elastic components (electrode array and tissue) compete to reach the lowest potential energy state. Accordingly, recommended actions could also include a pause or wait as a potential “action.” A pause of up to 10 seconds might be beneficial in some instances. The system could indicate to the surgeon that it is purposefully pausing (not broken down). Embodiments include recommending a pause of at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 75, 90 or 120 seconds or any value or range of values therebetween in 1 second increments.

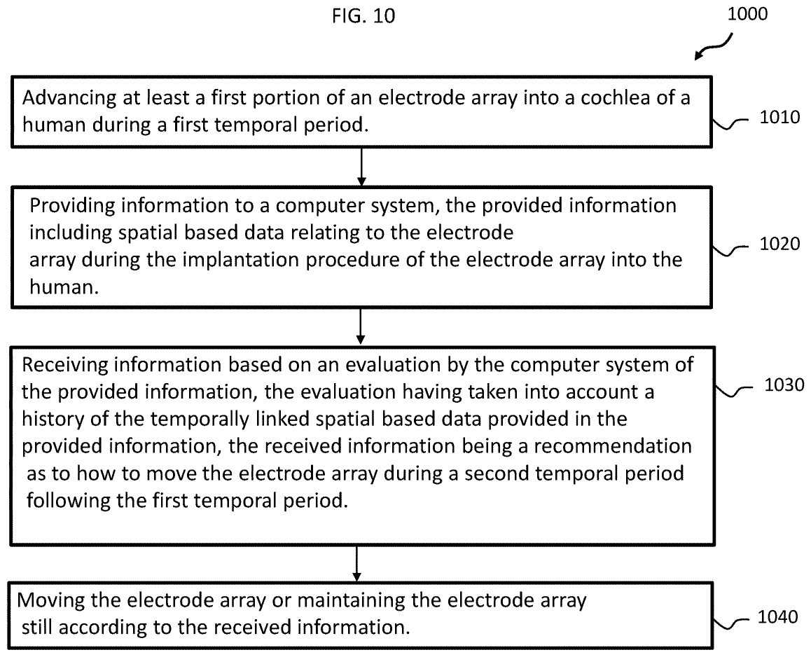

In view of the above, there is a method, such as that represented by the flowchart of FIG. 10, which comprises method action 1010, which includes the action of advancing at least a first portion of an electrode array into a cochlea of a human (a recipient) during a first temporal period. In an embodiment, this is part of an implantation procedure (e.g., a cochlear implant implantation procedure) done in a surgery room or a health care facility. This can correspond to inserting an electrode array partially or completely into a cochlea. In an embodiment, of the total length of the array to be inserted and that will be inserted upon completion of implantation, at least and/or equal to and/or more than ABC %, where ABC is 5, 10, 15, 20, 25, 30, 35, 40, 45, 50, 55, 60, 65, 70, 75, 80, 85, 90, 95, or 100 or any value or range of values therebetween in 1 increments, of the length of the array can be inserted into the cochlea by the point that method action 1010 is completed.

In an embodiment, the action of advancing corresponds to advancing the array by at least and/or equal to and/or more than ABC % of the length of the array. For example, method action 1010 can be executed by inserting less than 7% of the length of the array, which results in 67% of the array being inserted into the cochlea. (Here we are keying the amount of insertion during an insertion action to the entire length of the array. An amount that is 7% of the length of the array is inserted. That could be the first 7% or the last 7%, or anywhere in between. In this regard, the first temporal period can be the variable that defines the action. In this regard, the first temporal period can be a period that is less than, equal to or greater than DEF seconds, where DEF is 0.1, 0.2, 0.3, 0.4, 0.5, 0.6, 0.7, 0.8, 0.9, 1.0. 1.1, 1.2, 1.3, 1.4, 1.5. 1.6, 1.7, 1.8, 1.9, 2, 2.25, 2.5, 2.75, 3, 3.25, 3.5, 3.75, 4, 4.25, 4.5, 4.75, 5, 6, 6.5, 7, 7.5, 8, 8.5, 9, 9.5, 10, 11, 12, 13, 14, 15, 16, 17, 18, 19, 20, 25, 30, 35, 40, 45, or 50, or any value or range of values in 0.025 increments. The reason for the fine increments can be for the discretization of the movement(s) and/or positions of the array. In this regard, as noted above, a constant movement of the array can be broken down into discrete movements. Conversely, some scenarios of implantation can correspond to insertion of the array in a slow and/or methodical step-wise fashion, with some forward and backward movements and/or or pauses (intentional or unintentional). The teachings herein capture those movements, and convert them to numerical format, by, for example, receiving locational input using the devices disclosed herein and establishing a dataset of location vs. time and/or speed vs. time, and thus create a virtual “log” of the movements. This can be converted to an equation (curve fitting) or just be used as a discrete dataset. The time entries can be constant or can vary, providing that such has utilitarian value. Accordingly, there could be a dataset for the first temporal period where, for example, at 0.4 seconds, the insertion depth is 3 mm or X percent, at 0.6 seconds, the insertion depth is 3.4 mm or Y percent, etc. This is logged in some embodiments. And note that while the embodiments herein are often and typically described in terms of distance of insertion (e.g., 5 mm), embodiments can also be implemented where the amount of insertion is presented in terms of angular insertion amount. Any disclosure herein corresponding to a distance of insertion and/or withdrawal corresponds to an alternate disclosure of an angular amount of insertion and/or withdrawal amount. And embodiments include providing the instructions a recommendation in terms of angular amount as opposed to distance, etc.

Embodiments include insertion actions where the electrode array is inserted over a time period such as, for example, from first entry of the array into the opening into the cochlea to final placement of the electrode array into the cochlea and/or to the first entrance of “packing” into the opening (packing tissue around the array to seal the opening), the time is less than and/or equal to 10, 9, 9, 7, 6, 5, 4, 3, 2, or 1 minutes, or any value or range of values therebetween in 0.1 minute increments (e.g., 7.3 minutes, 3.4 minutes, 2.1 to 5.5 minutes, etc.). In an embodiment, the array is inserted in a step-wise fashion (e.g., no more than 10, 9, 8, 7, 6, 5, 4, 3, 2, or 1 mm, or any value or range of values therebetween in 1 mm increments within a 30, 25, 20, 15, 10, or 5 second intervals, or any value or range of values therebetween in 1 second increments).

Method 1000 includes method action 1020, which includes the action of providing information to a computer system, the provided information including spatial based data relating to the electrode array during the implantation procedure of the electrode array into the human. In an embodiment, this is done automatically. By way of example only and not by way of limitation, in an exemplary embodiment, there is a sensor on an electrode array insertion device, such as those detailed in one or more of the above noted publications, or otherwise an arrangement that can read or otherwise determine an amount of array and/or a speed and/or a rate that the array is inserted in the cochlea. The sensor can provide output to a computer via a wired or wireless transmission system. Because speed and/or rate of insertion has a spatial component, that is included within “spatially based.” Further, an imaging system or an arrangement that can determine a pose or otherwise an orientation of the electrode array can provide the information to this computer system. Some additional details of these arrangements are described below. In an embodiment, the action of providing information to the computer system can be done in an audible manner, such as, for example, the surgeon or healthcare professional “reading out” or otherwise declaring how much of the electrode array has been inserted in the cochlea. This can be done based on the surgeon's (any disclosure herein of a surgeon corresponds to an alternate embodiment where some other type of healthcare professional is implementing the action and/or vice versa, providing that the art enables such and such has utilitarian value) ability to “estimate” the amount of array that has been inserted into the cochlea with or without the aid of markers on the electrode array. In an embodiment, the array could have indicators that can provide an indication as to how far the array has been inserted, such as an indicator that is present every three or five mm, and/or in an exemplary embodiment, the surgeon can count electrodes that have been inserted into the cochlea or otherwise can no longer be seen and/or otherwise still can be seen, and then subtracted from the total electrodes (e.g., if seven electrodes can be seen, the surgeon could call out seven and the computer system could determine that 15 electrodes have been inserted because the electrode array is a 22 electrode contact electrode array). And in this regard, an aide or a nurse or a helper could type in the information from the surgeon. In an embodiment, there is a computer system that includes a microphone that captures sound, and this microphone has a voice to text capability or voice to digital capability that can deduce what the surgeon is saying and convert it to numerical format or a computer readable format. In any event, the action of talking into the speaker and the speaker transducing the sound into an electronic signal for example, which signal is provided to the computer, corresponds to providing information to a computer system. Any device, system, and/or method that can enable the provided information to be provided to a computer can be utilized in at least some exemplary embodiments.

In an embodiment, the provided information to the computer includes temporally linked spatial based data relating to the electrode array during implantation procedure of the array. In this regard, the information could be a distance correlated to a time data set. By way of example only and not by way of limitation, in an embodiment, the aforementioned insertion device could have a computer system or a subsystem of its own with a timer where the output is correlated to time. Also, this could also be computed by a smart implant if powered up during surgery (such as for a totally implantable cochlear implant or a conventional implant normally powered from an external coil). Either could have a radio link to a smart phone or tablet or some other computing device in the operating room, for example. Computing could also be done in a cloud based system for example. (Any computational actions herein can be done in a could based system providing that the art enable such.)

The output could be data packets having distance correlated to time which is provided to the computer system. Conversely, in some embodiments, it is the computer system that correlates the input which can be spatial input only, with time. This could be more utilitarian with respect to the devices and systems that utilize automated sensors (where there is little to no time lag between the recording at the sensor and the receipt of the electrical signal or data set at the computer system in an exemplary embodiment) as opposed to the aforementioned verbal calling out of distance. In an embodiment, method action 1020 is executed in real time with the insertion process/the implantation process. In an embodiment, receipt by the computer system occurs within 5, 4, 3, 2, or 1 or less seconds from the actual spatial feature of the electrode array (that is, if the array is inserted 3.7 mm, the computer system receives the data corresponding to such within for example, two seconds).

In an embodiment, the action of method action 1020 can correspond to providing data over a server or over a cell phone connection or over a telephone connection, landline or otherwise, to a remote computer system. In an embodiment, the computer system can be at least tens or hundreds or thousands of miles away from the actor who is executing method action 1020. In an exemplary embodiment, the actor of method action 1020 acts to execute the method action by inputting this data into a local computing device, where that computing device can transfer the data or otherwise provide data based on the data that is inputted into the local computing device to the computer system. That said, in an exemplary embodiment, the computer system can be located in the surgical room where the implantation process is being executed, or, can be located in another room of the hospital for example.

As will be described in greater detail below, the computer system is configured, in at least some embodiments, to evaluate the provided information or otherwise analyze the provided information for utilitarian effect. In this regard, in an exemplary embodiment, method 1000 includes the method action 1030, which includes the action of receiving information based on an evaluation by the computer system of the provided information, the evaluation having taken into account a history of the spatial based data (temporally linked or otherwise) provided in the provided information, the received information being a recommendation as to how to move the electrode array during a second temporal period following the first temporal period. In an exemplary embodiment, the second temporal period can extend immediately and be contiguous with the first temporal period, or there can be one or two or more temporal periods between the first temporal period in the second temporal period. In an embodiment, the second temporal period can be a period having a length corresponding to less than, equal to, or greater than DEF seconds, and note that the length need not be the same as the length of the first time period (the reference to DEF is used for textual economy). Exemplary embodiments associated with the recommendations and types of recommendations will be described in greater detail below, but briefly, in an exemplary embodiment, the recommendation could be to stop inserting the electrode array forward.

Method 1000 further includes method action 1040, which entails moving the electrode array or maintaining the electrode array still according to the received information. In the aforementioned example, this would be stopping movement, and thus maintaining the electrode array still, such as for example, by any of the pause times detailed herein. The movement could be controlled by a system of which the computer system is a part. For example, the computer system could be a subsystem of a robotic system, where the received information is received by a microprocessor that controls the actuator of the robot. However, before such movement is executed, the surgeon or healthcare professional would approve such, by affirmative action, consistent with the fact that method action 1030 is a recommendation based action. More on this below.

In an embodiment, the history includes the most recent movement being the insertion of the array further into the cochlea and the electrode array is almost fully inserted. That could be the action within any of DEF seconds of the first temporal period or a subset thereof. For example, the history could include the most recent two or three or four or five or six or seven movements. And note that the history could be, or can include a single continuous movement over a relatively lengthy period of time. This single continuous movement could be discretized as noted above. This would still be a single continuous movement. That said, the history could “ignore” or otherwise not include some movements. By way of example only and not by way of limitation, in some exemplary embodiments, there can be utilitarian value with respect to ignoring or otherwise not considering movements that occurred more than five seconds prior to a point in time with respect to a continuous movement. In this regard, the movement prior to five seconds could be, statistically speaking, irrelevant with respect to the utilitarian value of analyzing such. Conversely, the entire history could be taken into account.

In an embodiment, the history includes that the array was stationary, and a pose of the array was that the electrode array is clinically against a modiolus wall of the cochlea. By “clinically against,” it is meant that the array is sufficiently close to the modiolus that for all practical purposes, it would be considered against the modiolus wall of the cochlea with respect to imaging or otherwise with respect to performing an analysis of the effects of the cochlear implant electrode array. Thus, “clinically against” includes in direct contact with the wall and also offset by a de minimus amount.

In an exemplary embodiment, the received information is to move the electrode array further into the cochlea. In an embodiment, the received information could be the amounts to move the electrode array further into the cochlea. In this regard, in an exemplary embodiment, the received information could indicate to move the electrode array by an amount or less than 15, 10, 9, 8, 7, 6, 5, 4, 3, 2, or 1 mm, or any value or range of values therebetween in a half millimeter increment. And note that this could be preference by the qualifier “about.” The exact measurements will not be utilized in an exemplary embodiment where the surgeon is manually inserting the electrode array. Conversely, exact amounts could be directed towards an arrangement where the actuator of the robot is moving the array.

The received information could be to not move the electrode array or otherwise stop moving the electrode array. This could be because the electrode array is fully inserted into the cochlea for example. This could also be because the history indicates that the prior movement forward caused the electrode array to move to a location where the electrode array was no longer clinically against the modiolus wall. Accordingly, the computer system could determine that based on the history, further movement will cause the array to move away from the wall, which in some embodiments is not desired (for example, some cochlea are obstructed by growths or other anatomical abnormalities, and there are few ways around this, and the computer will make a determination that as a matter of statistics, this is the best positioning that can be achieved, even thought the array is not as far into the cochlea as would be desired), and therefore, the system will recommend that no further insertion be implemented.

Accordingly, in an embodiment of method 1000, the history includes the most recent movement being the insertion of the array further into the cochlea (by whatever amount) and the electrode array is almost fully inserted. Further, in an exemplary embodiment, the received information is also based on an evaluation by the computer system of the provided information, the evaluation having taken into account current spatial based data of the array. This is distinguished from, for example, history. In the context as used herein, the phrase history does not include current status. But note that the current status could be the same as a historical status such as where, for example, the electrode array has not moved since “historical times.” In an exemplary embodiment, a current pose is that a mid-section of the electrode array is clinically away from a modiolus wall of the cochlea. And in this method, in an exemplary embodiment, the received information is to move the electrode array backwards. This is done, because, based on the algorithm or otherwise the lookup table of the computer system, in these situations, based on the historical data and the current pose, there has been utilitarian value in the past with respect to withdrawing the electrode array a certain amount or otherwise pulling the electrode array backwards. The innovations associated with developing this database or otherwise a model that is utilized by the computer system will be described below.

In an embodiment, the current pose can be that of the portions of the electrode array that are away from the modiolus wall (not just clinically away) the average distance (mean, median, and/or mode) is less than, greater than and/or equal to 0.1, 0.125, 0.15, 0.175, 0.2, 0.25, 0.3, 0.35, 0.4, 0.45, 0.5, 0.6, 0.7, 0.8, 0.9, 1, 1.25, 1.5, 1.75 or 2 millimeters, or any value or range of values therebetween in 0.005 millimeter increments. In an embodiment, the algorithms or the like can be configured to determine that if the average distance is greater than and/or equal to any one of the just detailed values (e.g., 0.155 mm, 0.0170 mm, etc.), an action should be taken to move the electrode array closer to the wall (which could be to withdraw the array a certain amount) or stop inserting or otherwise that this is an unacceptable distance away from the wall. In an embodiment, the algorithms or the like can be configured to determine that if the average distance is less than and/or equal to any one of the just detailed values, an action should be taken to continue to move the electrode array forward or otherwise stop moving the electrode array forward or otherwise that this is an acceptable distance from the wall. Note that these values may or may not be “clinically against” values. It could be that even if the array is not clinically against the wall, that is acceptable. Indeed, it could be that the history indicates that it is impossible or otherwise not feasible to have the array clinically against the wall. In this regard, the contour of the modular wall varies from patient to patient and a pre-curved electrode array is designed for the average patient. Accordingly, there will be instances where the design of the electrode array will simply be incompatible with a certain patient because the design is for the average patient and this particular patient could deviate from the average person's anatomy by a sufficient amount which would otherwise prevent the array from being against the modiolus wall. Accordingly, the algorithms of the computer system or the models of the system could be configured to determine that this is good enough or otherwise the best that is going to be achieved under the circumstances, without taking some other action that might not be desirable. Again, more details of the algorithm will be described below.

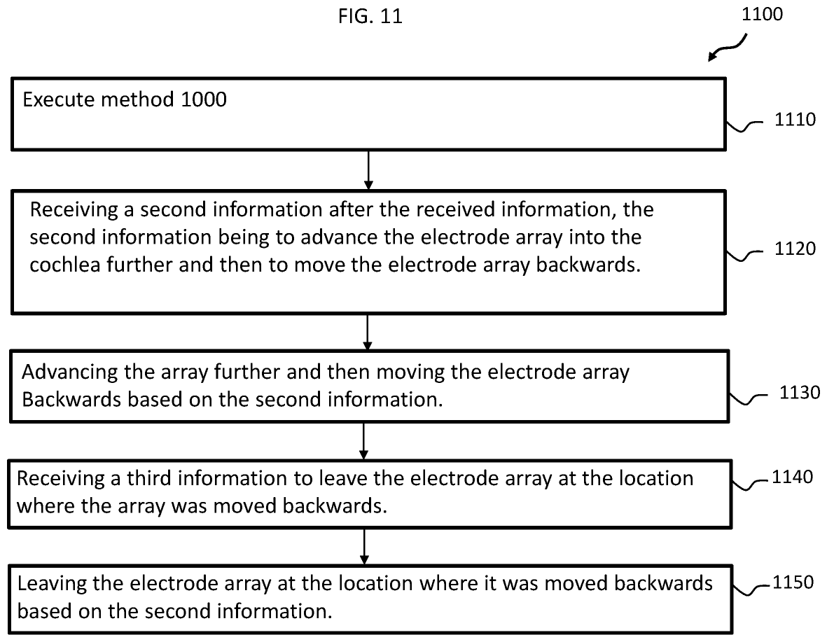

FIG. 11 presents another exemplary flowchart for an exemplary method, method 1100. Method action 1110 of method 1100 includes the action of executing method 1000. In an exemplary embodiment of this method, the method starts off where the history includes the most recent movement in the electrode array and the array is at least almost fully inserted, and the evaluation takes into account the current spatial based data of the array and the current pose is that a mid scaler section of the electrode array is clinically away from a modiolus wall of the cochlea. The received information was to move the electrode array backwards (last received information). Method 1100 includes method action 1120, which includes the action of receiving a second information after the received information, the second information being to advance the electrode array into the cochlea and then to move the electrode array backwards. In an exemplary embodiment, this second information can be received after method action 1040. In an embodiment, additional information can be provided to the computer system in between method action 1040 and method action 1120, such as, for example, providing information to the computer system including spatial features of the array associated with method action 1040, whether that be the movement of the electrode array and/or the pose that is currently the case, etc.

Method 1100 further includes method action 1130, which includes the action of advancing the array and then moving the electrode array backwards based on the second information. In this regard, in an exemplary embodiment, the computer system could analyze that there is utilitarian value with respect to moving the array as just noted. In an exemplary embodiment, the utilitarian value of this can be to determine whether or not the pose of the electrode array returns to that which was the case prior to the action of withdrawing the electrode array a bit, which was that the electrode array bowed away from the modiolus wall by an amount that was unacceptable. The action of withdrawing the electrode array a bit more could cause the action of moving the electrode array closer to the wall. Here, the movement forward and then backward is to “check” whether or not the array will again bow away from the wall with forward movement. In this exemplary scenario, that is the case—the array bows away from the wall, and then moves towards the wall/closer to the wall upon the movement of the array backwards.

Briefly, in an alternate exemplary embodiment, method action 1130 could instead include the action of moving the electrode array backwards and then advancing the electrode array based on the second information. This could have utilitarian value with respect to freeing up the electrode array if the tip, for example, is “stuck” or otherwise held up on a non-smooth surface of the modiolus wall of the cochlea.

Method 1100 also includes method action 1140, which includes receiving a third information to leave the electrode array at the location where it was moved backwards. In this regard, in an exemplary scenario, the computer system could determine based on the models and algorithms that is unlikely that the electrode array will be “freed” or otherwise have a geometry or pose that is closer to the modiolus wall than that which is the case at its current location in the retracted position. This determination can simply be based on statistical factors indicating that this is what should be done based on past occurrences with respect to other humans and other cochlear implantation procedures or comparison of the resting state of the array with the dimensions of the cochlea as determined from pre-operative imaging that were utilized to construct the model and/or algorithm, again as will be described in greater detail below. In between the action of moving the electrode array forward and moving the electrode array backward, the computer system can analyze additional information provided thereto to determine a pose for example of the array when the electrode array was moved forward. If the electrode array bows away from the modiolus wall for example, this would be an indication that the array should be pulled backwards, hence the instruction to do so or otherwise the recommendation to do so. Method 1100 further includes method action 1150, which includes the action of leaving the electrode array at the location where it was moved backwards based on the second information. Here, the electrode array is not “fully inserted” with respect to the standard procedure of cochlear implant electrode array implantation. In this exemplary embodiment, there can be one or more electrodes that are not located inside the cavity of the cochlea (these one or more electrodes could be located in the middle ear cavity, or located in the passageway into the cochlea) and/or otherwise one or more electrodes that are not “as far into the cochlea” as would be the case with respect to standard cochlear implantation procedures. In an exemplary embodiment, the “channels” for the electrode array would be adjusted to take into account the fact that not all electrodes are usable in the normal manner, if at all (because one or more electrodes are located outside the cochlea are in the passageway to the cochlea). The idea is that there is more utilitarian value with respect to having the electrode array as close to the modiolus wall as possible even though one or more channels are not usable or less usable than that which would otherwise be the case versus having all channels available but having the electrode array away further away from the wall. In an exemplary embodiment, the scenario of implantation results in the average distance from the wall with respect to the sections that are away from the wall being reduced by ABC %.

In an exemplary embodiment, the history includes the most recent movement being to insert the array further into the cochlea and the electrode array is less than ABC % of the way fully inserted (e.g., ¾ths, ⅔rds, ½, ⅓^{rd}, etc.) and the current pose is that an apical section of the electrode array is clinically away from a modiolus wall of the cochlea. Here, the received information is to move the electrode array backwards by a relatively significant amount, and then move the electrode array forward by more than an amount corresponding to the relatively significant amount. In an embodiment, the received information is to move the electrode array backwards by a specified amount, and then move the electrode array forward by more than an amount corresponding to the specified amount, wherein the specified amount is less than, greater than and/or equal to ABC% of the planned insertion depth of the array and/or the distance spanned by the electrodes and/or the carrier member of the array and/or GHI mm, where GHI is 1, 2, 3, 4, 5, 6, 7, 8, 9, 10, 11, 12, 13, 14, 15, 16, 17, 18, 19, 20, 21, 22, 23, 24, or 25 or more, or any value or range of values therebetween in 0.25 mm increments.

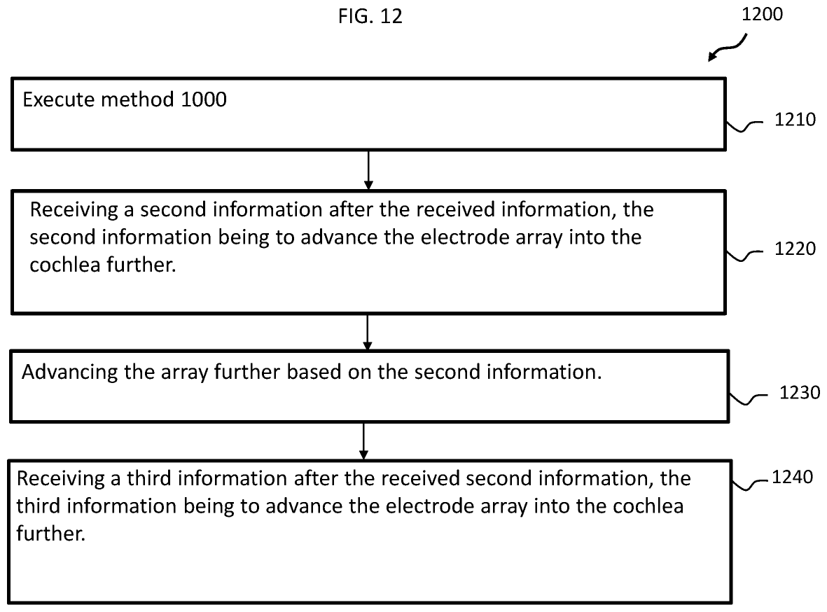

In an embodiment, after the above-noted backwards-forwards movement, there is a method 1200 represented in part by the flowchart of FIG. 12, where there is method action 1210, which includes executing method 1000 (other actions can be taken as well). Method 1200 includes method action 1220, which includes receiving a second information after the received information, the second information being to advance the electrode array into the cochlea further. This could come with a specified amount, or can be in terms of an instruction to keep and searching the electrode array. Method 1200 further includes method action 1230, which includes the action of advancing the array further based on the second information. Method 1200 also includes method action 1240, which includes the action of receiving a third information after the received second information, the third information being to advance the electrode array into the cochlea further. In an exemplary embodiment, the method includes the action of advancing the electrode array further. In an exemplary embodiment of method 1200, the array was determined to have bowed away from the modiolus wall. However, the action of moving the electrode array backwards by the specified amount or by the significant amount freed up the electrode array from that which the tip was hung up, and then the subsequent forward movement moved the tip past that point where the tip was previously hung up. The computer system thus deemed that the electrode array could be moved further into the cochlea without at least increasing the deleterious bowing of the electrode array from the modiolus wall. And note that in some embodiments, it can be that the electrode array has bowed away from the modiolus wall. It is just that the electrode array does not bow further away from the wall with forward movement, and thus a determination is made that the bowing, while irritating or otherwise potentially problematic, is something that must be accepted, at least if the electrode array could be moved further into the cochlea without increasing the distance from the wall of the array, or at least not by a significant amount.

In an exemplary embodiment, upon an action to reduce or otherwise eliminate the separation of the electrode array from the modiolus wall and/or a determination that the electrode array has moved away from the modiolus wall by a certain amount, such as by an amount that renders the array no longer clinically against the modiolus wall, and/or by an amount specified, such as for portions of the electrode array that are away from the modiolus wall (not just clinically away), the average distance (mean, median and/or mode) is less than, greater than and/or equal to 0.1, 0.125, 0.15, 0.175, 0.2, 0.25, 0.3, 0.35, 0.4, 0.45, 0.5, 0.6, 0.7, 0.8, 0.9, 1, 1.25, 1.5, 1.75 or 2 millimeters or any value or range of values therebetween in 0.005 millimeter increments, further advancement of the electrode array does not increase the average (mean, median and/or mode) separation distance by more than and/or equal to JKL %, where JKL is 5, 10, 15, 20, 25, 30, 40, 50, 60, 70, 80, 90, 100, 125, 150, 175, 200, 225, 250 or any value or range of values therebetween in 1% increments. In an embodiment, the computer system estimates or otherwise predicts that this will be the case, and then recommends the further movement into the cochlea of the electrode array.

In an embodiment, the action of moving the electrode array according to the received information is executed using an automated insertion component automatically controlled based on the received information. Additional details of this will be described below, but, briefly, in an exemplary embodiment, there can be an automated handheld actuation device such as an electrode guide with rollers that advance and/or retract the electrode array into the cochlea based on a received signal. This handheld actuation device can be handheld by a surgeon or other healthcare professional during the surgery. In an exemplary embodiment, the actuation device operates based on control signals from the computer system or of the computer system, but is subject to actuation only upon the approval of the surgeon. In other embodiments, the actuation device is controlled by the surgeon, such as with a toggle switch or the like, where the surgeon controls the actuation based on the received information.

In an embodiment, the provided information includes, in one or more data transfer periods to the computer system, LMN movement(s), past and/or present, of the electrode array and/or at least LMN pose(s), past and/or present, of the electrode array over the period of time, and/or at least LMN status(es), past and/or present, of the electrode array, all over a period of time of at least DEF seconds, wherein LMN is 1 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 125, 150, 175, 200, 225, 250 275, or 300, or any value or range of values therebetween in 1 increment.

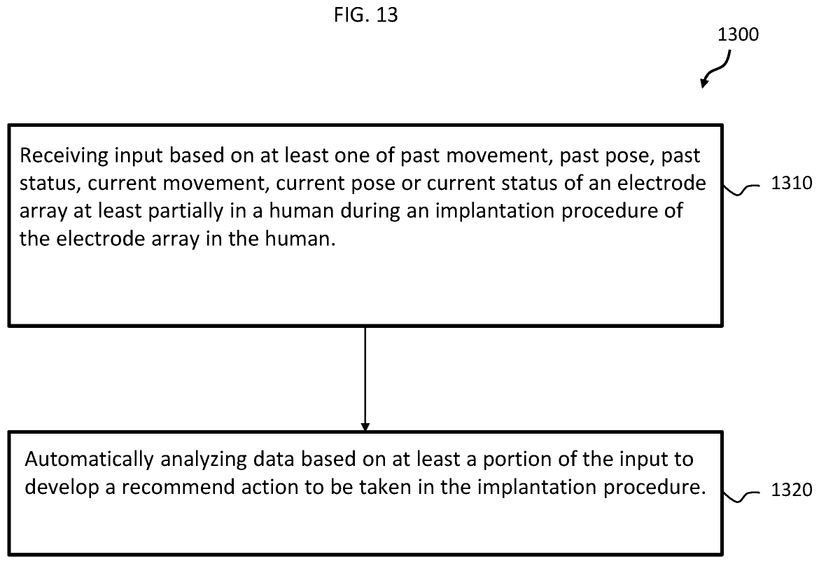

FIG. 13 shows another flowchart for another exemplary method, method 1300, which includes method action 1310, corresponding to receiving input based on at least one of past movement, past pose, past status, current movement, current pose or current status of an electrode array at least partially in a human during an implantation procedure of the electrode array in the human. Method 1300 also includes method action 1320, which includes automatically analyzing data based on at least a portion of the input to develop a recommend action to be taken in the implantation procedure. In this embodiment, the analyzed data includes at least one of:
current movement of the electrode array;
current pose of the electrode array; or
current status of the electrode array; and
   at least one of:
at least one movement of the array during a first temporal period prior to temporal current;
at least one pose of the array during the first temporal period; or
at least one status of the electrode array during the first temporal period.

Method 1300 can be executed by the computer system noted above or otherwise described herein. This could be a remote computer system remote from the surgical facility where the implantation procedure is taking place, as noted above, or could be located in the operating room or in the facility where the operating room exists.

With respect to method action 1310, the input can be a data transfer to the computer system and/or recalled from storage in a smart implant/smart external component. This can be done in real time with no time delay, or with a time delay relative to the features of the input at issue (hence the qualifiers “past” with respect to past movement, past pose and past status).

With respect to method action 1310, the input can be a data transfer to the computer system. This can be done in real time with no time delay, or with a time delay relative to the features of the input at issue (hence the qualifiers “past” with respect to past movement, past pose and past status). In an exemplary embodiment, the time period between receiving the input and the time that the feature was captured is less than equal to and/or greater than 10, 9, 8, 7, 6, 5, 4, 3, 2.5, 2, 1.5, 1, 0.75, 0.5, 0.25, 0.1, 0.05 seconds, or any value or range of values therebetween in 0.01 second increments. Note that in some embodiments, the past features can be updated to features already provided. In this regard, with respect to the embodiments that utilize the history of the spatial data associated with the array, there can be scenarios where the features provided to the computer system might not be current or otherwise in real time. Some features could be provided in real time, such as the current pose, or current movements. However, to execute an analysis utilizing the history of the spatial data relating to the array, the history need not be provided in a manner where the features are presented to the computer system in real time. In an exemplary scenario, the first time that input is provided to the computer system or otherwise received by the computer system could be upon a determination that the electrode array has moved away from the modiolus wall. The data transfer initiation could be based on some event that will always occur (as opposed to the aforementioned movement away from the wall). For example, the data transfer initiation could be initiated based on the distance that the array has been inserted into the cochlea, or an event such as the array answering the first turn of the cochlea, etc. The point is that method action 1310 is presented in terms of a flexible approach to receiving the data.

It is briefly noted that the spatial data associated with the current temporal period can correspond to the current movement, current pose and/or current status of method action 1310. But note these could also correspond to at least the past pose and/or past status of method action 1310. The received input need not necessarily be for example, the current pose of the electrode array. Note that method action 1310 requires only receiving input based on for example, the current pose. In this regard, if the input is an indication that the array has not moved since the past pose, and data based on the past pose was received, the current pose could be determined. It is also noted that the spatial data associated with the first temporal period prior to temporal current (current time) can correspond to the past movement, past pose and/or past status.

The movements of method 1300 can be defined with various temporal limits. For example, the past movement can be movement over a period of time such as a period of time lasting a tenth of a second, or period of time lasting a few seconds, by way of example only. And while the embodiments herein often focus on “repositioning” the array, which includes movements within the cochlea, or the more drastic concept of withdrawing the array completely and reinserting, embodiments can also include using a new array entirely (and a system that recommends such based on the data). In this regard, sometimes an electrode array becomes unusable (for a variety of reasons this can be the case) and it would be utilitarian for the surgeon to use another device (e.g., a backup device). The system could recommend such based on the data, and the system could also keep the history of the first device which failed to be inserted (or was not inserted) and refer to that history to advise on the insertion of a second device. The second device could be a different electrode array model (e.g., a straight array instead of a curved array, or another model of curved array, or visa-versa). The history could be collected in a traditional manner, such as by surgeons inputting data into a database or otherwise providing data to a central location where it is compiled, etc., or otherwise uploading the data to the cloud where it is compiled, consistent with the other data that is collected to establish the algorithms or otherwise train the neural network, or otherwise establish the statistical big data protocols, etc., Or the system could automatically log this and then update its own records or otherwise update the central database, etc. In some instances, sometimes the cochlea is obstructed somewhere beyond the visibility of the surgical microscope. In attempting to insert an electrode array to the full depth in such a scenario, the electrode array could become damaged and/or could damage the tissue of the cochlea. It could simply be that as a matter of statistics, it is essentially impossible or otherwise extremely unlikely that the array will ever be seated within the cochlea in a proper manner or otherwise in a manner that does not damage the tissue or the array with that particular electrode array. A second electrode array which could be the same or different type of electrode array, say a straight array instead of a curved array, or visa-versa, or a round cross-section array vs. a rectangular cross section array, or a styletted array vs. a styletless array, and all visa-versa, etc. Knowing the point of the apparent obstruction from the first insertion (based on latent variables, such as the insertion depth/insertion angle), the system could advise to slow down at that point and not push too hard or too far. If the second electrode array cannot get past the obstruction (or be further inserted than the first), the surgeon has the choice to leave the electrode array partly inserted, or perhaps to remove that electrode array before it is damaged and use a Contour depth gauge to open up the cochlea at the point of obstruction, or to take other actions that are typical solutions in the specific scenario. In an embodiment, the system can recommend one or more of these actions, and also this data can be collected and utilized to train the DNN and, etc., consistent with the teachings detailed herein. And all of this data can be collected/used during the insertion process to make the recommendations/forecasts.

In an exemplary embodiment, the analyzed data can include the current status of the electrode array, such as completely inserted into the cochlea or half inserted into the cochlea, or 7 mm thereof inserted into the cochlea, etc. The analyzed data can also include the past status of the electrode array. This can have utilitarian value with respect to the overall analysis by the computer system such as by linking the past status of the electrode array with a past pose of the electrode array, where the pairing can be utilized to more accurately predict how the electrode array might move in the future. By way of example, if the status of the array is only 1 or 2 mm inserted into the cochlea, and the pose is that there is a space between the modiolus wall and the electrode array, the system could determine that this is not a problem or otherwise should be ignored because this is what happens during the initial insertion process at least in some instances.

In an exemplary embodiment, the first temporal period extends a period of at least DEF seconds. The analyzed data can include less than, greater than, and/or equal to GHI discretized movements of the electrode array. In an exemplary embodiment, the input of method action 1310 is based on at least one of past movement or current movement, and at least one of the movements of the at least one of the past movement or current movement is continuous for at least DEF seconds, and the method further includes discretizing the continuous movement into the GHI discretized movements. In an embodiment, the analyzed data includes at least GHI poses of the array during the first temporal period, and/or the analyzed data includes the temporal current pose of the array. In an embodiment, the analyzed data includes the at least GHI movements of the array during the first temporal period and/or the analyzed data includes the temporal current movement of the electrode array. In an embodiment, the analyzed data includes at least GHI statuses of the electrode array during the first temporal period and analyzed data includes the temporal current status of the electrode array. In an embodiment, the analyzed data can also include a trajectory of the lead outside the cochlea if based on a robotic insertion method that can accurately measure and record or otherwise obtain data based on the relevant vector. For example only, it might be possible to estimate that the cochlea is rotated and/or the round window (in the case of a round window insertion) or other opening into the cochlea is oblique. This can tend to direct the electrode array tip (and sheath) straight at the modiolar wall in some instances. The system could advise to extend the round window opening and/or modify the cochleostomy to create a more favorable vector for the sheath or electrode outside the cochlea. Embodiments thus include collecting the data and utilizing this to train the neural network or otherwise establish the big data protocols and algorithms, etc., and also include obtaining data relating to the trajectories and vectors and utilizing this data to make the recommendations, etc., where the recommendations can include adjusting the angle or otherwise making a modification to the opening into the cochlea.

In an embodiment, the analyzed data includes at least GHI discretized movements based on movement(s) during the first temporal period, the analyzed data includes at least GHI poses of the array during the first temporal period (and the values need not be the same—the variable GHI is used for textual economy). In an embodiment, the analyzed data includes at least GHI statuses of the array during the first temporal period. And note that the discretized movements can correspond to individual movements of the array made by the surgeon or the actuator. By way of example only and not by way of limitation, in an exemplary embodiment, the actuator that is utilized to insert the array moved to the array in a stepwise fashion, such as, for example, one ¼^{th} of a millimeter per second, or ½ of a millimeter or 1 mm for example. In an embodiment, the movement takes place within ¼ or ½ a second for example. The remainder of the time constitutes stationary time for the electrode array, at least relative to the insertion device/actuator or the cochlea. Corollary to this is that in at least some exemplary embodiments, the surgeon when hand driving the electrode array (e.g., the surgeon is pushing on the electrode array to drive the electrode array into the cochlea), could push the electrode array or otherwise move the electrode array two or three or four mm every two or three or four seconds for example. The movement could take place within a second or half a second. The remainder of the time is stationary time. And note that the time periods can be variable and can be different from one segment/step versus another. The point is that the discretization can be that which corresponds to real life discretization of the movements and/or can correspond to a division of a continuous movement into segments.

The concept of discretization can also be extended to the other spatial features, such as, for example, the pose and/or the status. The discretization may be the same pose for a number of the discretization, as is the case for the movement for that matter. Accordingly, in an exemplary scenario, the insertion process can be discretized into tens or hundreds or thousands, tens of thousands or more “frames.” In an embodiment, any value or range of values of discretizations from 1 to 100,000 or more in 1 increment can be used in at least some exemplary embodiments.

In an embodiment, there is a system, such as the computer system detailed above, or another system, comprising an input suite configured to receive input based on a spatial feature of an implantable medical device during implantation into a human. This can be a server, or can be a USB sub-system, or can be a microphone or a video camera or still camera, consistent with the teachings above. In an embodiment, the input suite could be an insertion guide according to the teachings below, which insertion guide includes components that can determine the position of the electrode array. The input based on a spatial feature of the implantable medical device can be digital data or analog data, such as the output of the insertion guide, or can be an electrical signal or a magnetic signal, where the electrical signal corresponds to a change in voltage or current for example that results when an electrode of the electrode array passes by a contact. Other options can be, for example, the use of strain gauges within the electrode array or bragg gratings in an optical fibre in the electrode array that can provide information on the shape of the electrode array in real time or near real time. Any device, system, and/or method that can enable the system to receive input based on the spatial feature of the implantable medical device during implantation to be used in at least some exemplary embodiments.

The system also includes an output suite, which could be a server or a USB sub-system (where the input suite can be integrated with the output suite concomitant with the commonly implemented USB devices) or a speaker or a video monitor, etc. In an embodiment, the output suite could be the insertion guide according to the teachings below, which insertion guide includes components that move the array according to a received signal. The output of the output suite can be digital data or analog data, etc. Any device, system, and/or method that can enable the system to receive input based on the spatial feature of the implantable medical device during implantation to be used in at least some exemplary embodiments. The system also includes a data analysis computer in signal communication with the input suite and the output suite, the data analysis computer including circuitry and being configured to automatically analyze, at least in part with the circuitry, the received input as that input relates to a history of spatial features (e.g., movement, twist, pose, status, etc.) of the medical device during implantation and automatically develop, based on the analysis, a subsequent movement of the medical device to be implemented during implantation. The system is configured to at least one of automatically output data based on the developed movement via the output suite or move the medical device based on the developed movement via the output suite. With respect to the former, this could be via a speaker in the operating room that provides an audible statement as to the developed subsequent movement, where the surgeon or other healthcare professional can evaluate the merits of such and act accordingly. Also with respect to the former, this could be providing a control signal or the like to a robot or the actuator of the insertion guide to control the insertion guide to move the array accordingly. With respect to the latter, this could be completed by movements of the robotic actuator.

Accordingly, in an embodiment, the system includes and/or is in signal communication with a robotic device that is configured to robotically move the medical device during implantation.

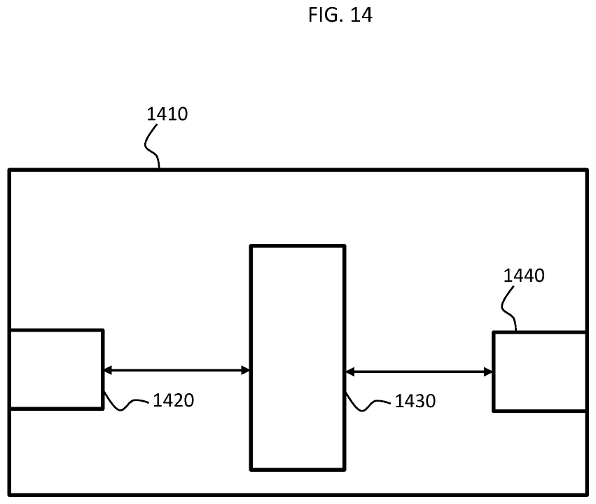

FIG. 14 shows the system 1410, with input suite 1420 and output suite 1440 in signal communication (e.g., by wires or by fiber optics, etc.) with the data analysis computer 1430.

Consistent with the teachings above, the data analysis computer can be configured to automatically analyze the received input also as that input relates also to a current spatial features (as opposed to a history—current is not history) of the medical device. Any of the spatial features detailed here can be used. In an embodiment, the history of spatial features includes a history of movements. In an embodiment, the history of spatial features includes a history of movements and a history of positions. The history can include any of the spatial features detailed herein.

In some embodiments, the system includes and/or is in signal communication with an automated monitoring device that is configured to automatically monitor movements and/or position of the medical device during implantation, wherein the received input is based on output from the automated monitoring device. Additional details of this will be provided below, but briefly, in an embodiment, an insertion guide, in the case of a cochlear implant electrode array, can be equipped with an “electrode counter” that can determine when an electrode of the array moves past a sensor, and thus determine the depth of insertion based on the electrode that is at/has passed the sensor. In an embodiment, there can be an arrangement that measures the impedance change as electrodes move into the inner ear. Because the electrodes go from air to perilymph, one can detect a change in impedance from high to low impedance. This can be used to determine the insertion depth, or otherwise to “count electrodes.” This can be done with an implant that is configured to measure the impedance change, or the outright impedance for that matter.

Again, more on this below. Moreover, again by way of example where the medical device is a cochlear implant electrode array, in an exemplary embodiment, the system is configured to discretize the input data to establish discretized movement and/or positions of the array (as detailed above). Additionally, the system can be configured to use the discretized input data to establish the history of the spatial features of the medical device.

The data analysis computer can be implemented via a wide variety of regimes. In an embodiment, the data analysis computer includes electronics and/or software that is a product of and/or resulting from machine learning that is configured to execute the automatic analysis. More specifically, at least some exemplary embodiments according to the teachings detailed herein utilize advanced learning processing techniques, which are able to be trained to detect higher order, and non-linear, statistical properties of data. An exemplary analytical technique is the so called deep neural network (DNN). At least some exemplary embodiments utilize a DNN (or any other advanced learning analytical technique) to analyze the historical spatial features and/or current spatial features and/or other data relating to the medical device during implantation. At least some exemplary embodiments entail training data analysis algorithms/developing models to detect subtle and/or not-so-subtle changes, and provide an estimate of future statuses and conditions, etc., and specific information thereabout, that can correspond to how the array will move/the future spatial features of the array. That is, some exemplary methods utilize learning algorithms such as DNNs or any other algorithm that can have utilitarian value where that would otherwise enable the teachings detailed herein to analyze the data relating to the array to predict how the array will move and/or to determine how the array should be moved to achieve a utilitarian placement, etc.

A “neural network” is a specific type of machine learning system. Any disclosure herein of the species “neural network” constitutes a disclosure of the genus of a “machine learning system.” Moreover, any disclosure herein of the species “machine learning” constitutes a disclosure of the genus of “artificial intelligence.” While embodiments herein focus on the species of a neural network, it is noted that other embodiments can utilize other species of machine learning systems accordingly, or the broader genuses noted, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Some of the specifics of the DNN utilized in some embodiments will be described below, including some exemplary processes to train such DNN. First, however, some of the exemplary methods of utilizing such a DNN (or any other system that can have utilitarian value) will be described.

It is noted that in at least some exemplary embodiments, the DNN or the product from machine learning, etc., or the results thereof, etc., is utilized to achieve a given functionality as detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product or results from machine learning, etc., that when used, results in that functionality, unless otherwise noted or unless the art does not enable such.

Additional details of the DNN and the training thereof are provided below.

In an embodiment, by way of example only and not by limitation, the medical device is a cochlear implant electrode array, the data analysis computer is an artificial intelligence subsystem, which can be a neural network, such as a deep neural network.

The system of which the subsystem is a part can be configured to predict, with the use of the artificial intelligence subsystem, a next behavior of the array based on the input. The system of which the subsystem is a part can be configured to determine, with the use of the artificial intelligence subsystem, the next movement of the array that should be taken based on the input. (The subsystem can have “prediction model(s)” and/or “determination model(s)” that can be used to make the determinations and/or predictions. More on this below.) In this regard, different systems, which systems are not mutually exclusive, can be utilized for different purposes. On the one hand, the prediction of how the electrode array will move given the past history and/or the current features of the array can have utilitarian value with respect to providing information to the surgeon or other healthcare professional that will enable him or her to decide what action should be next taken, which could be to obtain the predicted next behavior, or to avoid the predicted next behavior. For example, if the current spatial features of the array are such that the spatial features indicate that a tip fold over has occurred, a prediction can be made that continued insertion will further exasperate the tip fold over condition. In this instance, a plausible scenario is that the surgeon will not further insert the electrode array upon notification by the system that this is the case. But note that this prediction can also be utilized by the system to determine that another action should be taken. In this regard, the artificial intelligence subsystem could not only predict the next behavior, but also could determine what action should be taken based on that prediction. That is, the artificial intelligence subsystem could stand in the place of the surgeon or the healthcare professional. Note that in an alternate embodiment, it can be another artificial intelligence subsystem that takes the prediction and determines what is to be done in the next movement. That said, it need not be in artificial intelligence subsystem. In an exemplary embodiment, the system includes a number of preordained actions that should be taken based on various predictions. For example, in the after mentioned example, if the prediction is further exasperation of the tip fold over, a lookup table can exist in the system where for that prediction, there is the action of withdrawing the electrode array by a certain amount. And note that this can be in terms of degrees, such as if the tip fold over condition is more severe than other conditions, more withdrawal of the array could be in order, and thus the lookup table could have different distances for withdrawal correlated to different tip fold over conditions. And it is briefly noted that while this embodiment utilizes an artificial intelligence subsystem to make the prediction, in other embodiments, “big data” can be utilized to make the prediction. More on this below.

But note that instead of making a prediction and/or in addition to making a prediction, the system can determine the next movement of the array that should be taken based on the input. In this regard, the system need not necessarily know or otherwise “understand” or make a determination of what will happen with respect to the next behavior of the array. In artificial intelligence subsystem can be used to determine what should be done. By rough analogy, a child may not know what will happen if he or she puts a key into the hot portion of an electrical outlet. Electricity and the concepts thereof may be completely alien and unknown to the child. However, the child can know not to put the key in the hot portion. Conversely, the child can know to put the key into a door to unlock the door.

Returning back to the prediction and/or determinations made by the artificial intelligence subsystem, embodiments can include providing the prediction and/or providing the determination to the surgeon in a manner concomitant with the teachings above. Embodiments can also include utilizing the determination at least in an automated manner to control a robot or the like.

The predictions and/or determinations can be based on a series of scenarios of future actions. That is, the predictions and/or determinations can include if-then-else logic, and indeed, the algorithms used herein can utilize such to implement any one or more of the functionalities detailed herein. In if-then-else algorithm can be utilized to implement the data analysis computer and/or the artificial intelligence subsystems, providing that sufficient data is provided (more on this below). For example, the prediction of the next behavior of the array can be caveated with a given future movement. For example, if the prediction is an exasperated tip fold over, that prediction can be caveated with the action of moving the electrode array further into the cochlea by three or four mm for example. This can also be based on the speed at which the array is moved forward. The tip fold over may be exasperated if the movement is over or within a period of a second or two. Conversely, that tip fold over may not be exasperated if the movement is over or within a period of five or 10 seconds, or otherwise if the surgeon pauses for a certain amount of time. In this regard, a series of predictions could be made, such as, for example, the exasperated tip fold over prediction and a prediction of reduced tip fold over with movement of the array backwards by a certain distance. A prediction could be made that all tip fold over would be eliminated/tip fold over would be resolved with movement of the array backwards by certain amount. A prediction can be made that all tip fold over would be eliminated if the array was moved backwards by less than the aforementioned certain amount followed by pause for a certain amount of time (there can be utilitarian value with respect to not withdrawing the electrode array by the certain amount—the idea being for example in some scenarios that backwards movement of the array should be limited as much as possible—that said, in a scenario, if the array is being withdrawn back into the sheath, it could be acceptable to pull the array all the way back into the sheath, but if the sheath has already been removed, there is then a point of withdrawal beyond which the electrode array cannot be moved forward again without reinserting the sheath. This could also be learnt by the system and be a utilitarian part of the dataset used for complicated insertion processes.

With respect to the modiolus wall locations, again, a prediction can be made that further insertion of a certain amount or at least a certain amount will result in an increase in the average distance of the array from the wall. A prediction could be made that while there is currently a clinically significant separation of the array from the wall, continued further advancement at a given rate will eliminate that separation or potentially reduce that separation to an amount that is acceptable.

And in an exemplary embodiment, any one or more of the aforementioned movements could be the determination made with the use of the artificial intelligence subsystem corresponding to the above-noted next movement of the array that should be taken based on the input. And again, a prediction of what will result need not necessarily exist. The artificial intelligence subsystem can be configured with sufficient training or otherwise with sufficient data to “know” what to do based on the past history and/or the current spatial factors. This determination can be provided to the surgeon as a recommendation and/or can be presented as a control data set that can be approved or overridden by the surgeon as noted above.

These concepts can also be used to predict a scala piercing scenario. For example, there can be cases where the sheath is sometimes inadvertently inserted into the scala vestibuli rather than scala timpani, or otherwise the electrode arrays being inserted therein. In some embodiments, the system could determine, based on the training, or based on a big data set, that based on the size of the specific cochlea into which the array is being inserted, the electrode array is adopting an abnormal pose. Based on this latent variable, the system could automatically identify this as a scala vestibuli insertion, and notify the surgeon of such and/or recommend that the insertion be restarted or otherwise that the insertion be aborted and a new introductory be commenced. In this regard, embodiments include utilizing data relating to the cochlea or the features of the person who is the subject of the given insertion to make the determinations about what is occurring or otherwise what is going on and/or make the forecasts and predictions. Here for example, based on the size of the cochlea, the resulting geometry of the electrode array can indicate where the array is within the cochlea, whereas if the size of the cochlea was not known, or at least some data based on the size of the cochlea, such as relative data, the resulting geometry might not be able to be utilized to indicate where the array is located.

For example, based on the input, a prediction can be made that certain actions will result in an electrode array piercing the duct into which it is being inserted. By way of example only and not by way of limitation, a severely bowed array could result in an upward force imparted thereon that drives the tip upwards towards the easily pierced basilar membrane. The artificial intelligence subsystem could deduce from the amount of bowing that this could happen or is likely to happen, and thus make a prediction of such. Consistent with the teachings above, this could be caveated with a qualification, such as the speed of which the array is being inserted and/or the amount of force that is being applied to the array. For example, an array that is being inserted very slowly or at least relatively slowly and/or with relatively minimal force, such as about the minimum force needed to continue insertion, will be less likely to pierce the basilar membrane under the bowed condition than that which might be the case with respect to a relatively faster and/or relatively higher insertion force. Accordingly, the predictions can be qualified based on the force and/or the speed. And in the exemplary embodiment where the system utilizes the prediction to make a determination as to the next movement of the array, the termination could be to move the array slower or insert the array with less force. And if, for example, the insertion of the array with less force results in the tip “hanging up” and otherwise the apical portion of the array not proceeding further into the cochlea (this could be predicted as well, indicating that a higher force is needed), the prediction could be to increase the force a certain amount or otherwise the determination could be such.

In an exemplary embodiment, the artificial intelligence subsystem is configured to predict any one or more next behaviors that have been seen in the past or otherwise upon which the subsystem has been trained depending on the input. In an exemplary embodiment, the artificial intelligence subsystem is configured to make a determination as to the next movement of the array that should be taken based on or corresponding to any one or more of the next movements seen in its training. Accordingly, depending on the training, any occurrence or scenario or next movement that is possible or is at least commonplace in a cochlear implant electrode array implantation process can be part of the prediction and/or the next movement determination.

And with respect to training, in an embodiment, the artificial intelligence subsystem is a neural network that is a partially taught neural network that is trainable with feedback provided through the input suite or another subsystem of the system. Additional information about the training is discussed below.

In an embodiment, there is an apparatus, comprising a medical device insertion device, such as, by way of example, a cochlear implant electrode array insertion device. As mentioned above, this can be a robotic actuator such as an actuator of insertion device or, in some other embodiments as will be described in greater detail below, a more expanded version of a robot. That said, the device need not be a robot/have an actuator. Not all embodiments include an actuator that moves the array. The device can be a recommendation/physician's assistant device that instructs/recommends actions to take, where the surgeon inserts the array by hand or inserts the array using an automated device not in signal communication with the device.

In an exemplary embodiment, this insertion device includes an input component configured to receive data based on data related to an insertion process of a cochlear implant electrode array, the received data being received in real time relative to the process. Additional details of this will be discussed below, but note that the input component can correspond to any of that disclosed herein for receiving data based on data relating to the insertion process. By data based on data, this can correspond to the raw data, or a transposed version or manipulated version of the data, or new data that is developed based on the raw data. In an exemplary embodiment, this could be the output from a sensor, and thus the raw data from the sensor. In an embodiment, this can be data that corresponds to an evaluation of what that role sensor data means, and hence data based on data.

In an exemplary embodiment, with respect to the device under discussion, the device can include an output component configured to provide output to a user regarding an action to take with respect to implanting the array in a human medical device, such as the array, in a human. In addition, or alternatively, the device can also include an actuator configured to move the array relative to the human in an automated manner. With respect to the former, the output component can correspond to the various embodiments of the aforementioned output suite noted above. With respect to the latter, this can correspond to the actuator briefly noted above in that which will be described in greater detail below.

In an embodiment, the insertion device at least one of (i) includes non-transitory logic or (ii) has access to non-transitory logic and/or results of analysis of the non-transitory logic. The former is part of the device, wherein the latter is not part of the device. This latter arrangement could have utilitarian value with respect to having the logic or otherwise the “brains” in a remote location, where the device accesses that remote logic via the Internet or the like. This can enable the logic to be controlled and otherwise supervised and managed by a single entity, which entity could be unrelated to the entity utilizing the insertion device. This can have utilitarian value with respect to quality control and, in at least some exemplary embodiments, constantly updating or otherwise retraining the product of machine learning if such corresponds to the logic. Thus, in an exemplary embodiment, the logic can be artificial intelligence logic, such as, for example, the results of the product of machine learning.

In an embodiment, the non-transitory logic identifies the action to take (the “at least one of” where the device has the output component) based on the received data, the received data being related to the insertion process including a prior action of the actuator if present (not all embodiments include an actuator that moves the array—again, the device can be a recommendation/physician's assistant device that instructs/recommends actions to take, where the surgeon inserts the array by hand or inserts the array using an automated device not in signal communication with the device) or another actuator (the another actuator could be separate from the device) configured to move the array in a manually controlled manner and/or a prior movement of the electrode array.

With regard to the first introduced actuator, the actuator can provide an indication of the movements and/or the status or other spatial features of the electrode array. If, for example, the prior action of the actuator is to advance the electrode array 3 mm, this data could be used by the logic to identify the action to take (e.g., the recommendation to the surgeon or to subsequently control the actuator, etc.). In this arrangement, the actuator provides a feedback or otherwise acts as the sensor. (There can be utilitarian value to an actuator that does not have precise measurement sensors that measure the distance, but instead use the latent variable of actuation to determine the distance of insertion.) With regard to the second introduced actuator, this could be a manually controlled actuator where the actuator is configured to output a signal indicative of the movements of the actuator, including prior movements, etc. (A manually controlled actuator could be an actuator that has an electrical motor/actuator that is controlled by the surgeon, such as via a toggle switch.) Alternatively, the input can be based on a verbal statement that the surgeon who has control of the actuator has advance the electrode array by 2 mm for example, and that would be inputted into the logic. With respect to the prior movement of the electrode array, this again could be the surgeon or other healthcare provider “calling out” the movement, which callout is recorded or otherwise captured by microphone, and converted to a numerical format that can be evaluated by the non-transitory logic. This could be from the insertion guide if used, which has sensors, etc.

Alternatively, and/or in addition to this, the logic controls the actuator based on the prior action of the actuator and/or a prior movement of the electrode array. The above noted explanations/examples are applicable here.

As seen from the above, the apparatus has various configurations, from a robotic device to an advisory device.

In an embodiment, the logic is configured to predict a future movement and/or position of the array based on a hypothetical future movement of the array from data based on at least one of a current position, a past position, a current movement or a past movement of the electrode array. In this embodiment, the output is based on the prediction. Note that the hypothetical future movement and the predicted future movement is not just a truism that if the array is moved according to X, the array will be moved according to X, always. It very well may be that the array moves accordingly, but it could be that the array moves differently. In other words, the array may not move the way that you want it to move or otherwise that you expect the array to move. In an exemplary embodiment, there are at least RJR past position(s) and RJR past movements, where RJR can be less than, greater than or equal to 1, 2, 3, 4, 5, 6, 7, 8, 9, 10, 11, 12, 13, 14, 15, 16, 17, 18, 19, 20, 21, 22, 23, 24, 25, 26, 27,2 8, 29, 30, 31, 32, 33, 34, 35, 36, 37, 38, 39, 40, 41, 42, 43, 44, 45, 46 47, 48, 49, 50, 55, 60, 65, 70, 75, 80, 90, 100, 125, 150, 175, or 200, or any value or range of values therebetween in 1 increment, and the values need not be the same for the position and the movement. This is consistent with the discretized movements/positions detailed above, but note also that this can be separate movements and/or positions, at least with respect to the lower numbers. The apparatus takes into account the history and/or the current position and/or movement to make a prediction about the future movements/positions.

In an exemplary embodiment, there can be RJR movements and/or positions predicted. In this regard, a dynamic future of the electrode array can be predicted. this can be placed on a video screen of the like and shown to the surgeon or other healthcare professional to visually demonstrate how the electrode array will move and/or the positions of the electrode array will take over time in the future.

In an embodiment, the output and/or the action is based on the prediction.

Corollary to the above is that the logic can be configured to determine a future movement based on a hypothetical future movement of the array from data based on at least one of a current position, a past position, a current movement or a past movement of the electrode array and the output and/or the action is based on the determination. In an embodiment, the determination can be provided to the surgeon as advice/recommendation, concomitant with the above.

The idea to these embodiments is that the history of the positions and/or movements, etc., of the array can be utilized to predict what will happen in the future, or otherwise determine what should be done with respect to having the array perform as desired in the future with respect to the position and insertion thereof.

Embodiments have focused on, at least in part, artificial intelligence or the like and otherwise machine learning. But embodiments can utilize big data. In an exemplary embodiment, the apparatus at least one of includes or has access to a database of array behaviors, the behaviors being behaviors during implantation of the array into a cochlea. These can be based on prior insertions for other people, as noted herein. In an embodiment, the database has at least 2, 3, 4, 5, 6, 7, 8, 9, 10, 15, 20, 25, 30, 35 or 40 or more or any value or range of values therebetween in 1 increment times the data, on an “insertion procedure basis,” than that used to “train” the machine learning embodiments. In an embodiment, the logic is configured to automatically query the database based on the received data to identify an array behavior corresponding to a behavior in the received data, and provide the output and/or control the actuator based on the identified array behavior.

In an embodiment, the logic includes one or more of a prediction model that automatically predicts a future movement behavior of the array or a cohort comparator that automatically identifies a cohort of movement behaviors of the array with statistically significantly similar and/or the same movement and/or positional scenarios as the array. In this embodiment, the logic uses the model and/or comparator to identify the action to take and/or to move the actuator in the automated manner.

More specifically, with the availability of large datasets (i.e., big data) with many subjects'/implantations' records (for example, more than the threshold needed to develop a utilitarian prediction model using the AI/machine learning, but not necessarily more than what such model uses or could use, because the model can get better potentially with more records, but it could work satisfactorily with fewer records), it is possible to directly compare, for example, insertion processes, with a cohort of insertions that are similar and/or have the same factors. The cohort is generated when a cohort comparator is run rather than being predetermined. Thus, embodiments include the establishment of a bespoke cohort of insertion procedures. Thus, in an embodiment, a bespoke cohort of people is developed for each new implantation. Based on a set of utilitarian preoperative/pretreatment factors (e.g., age, duration of ailment (e.g., hearing loss or sight loss or balance loss), aetiology, clinical measures, etc., and/or movements and/or any of the spatial features detailed herein, filters are constructed by the device, automatically, to identify and isolate a cohort of statistically similar people or insertion procedures. Based on this data, the cohort can be identified and the movements of the array during the insertion process can be determined and/or the spatial features can be predicted. In an embodiment, the logic includes the prediction model, and/or the logic includes the electronics includes the cohort comparator. In an embodiment, the apparatus includes the model and the model is a product of and/or resulting from machine learning that is used by the apparatus to predict movement behavior and/or future positional locations of the array based on the input into the input suite.

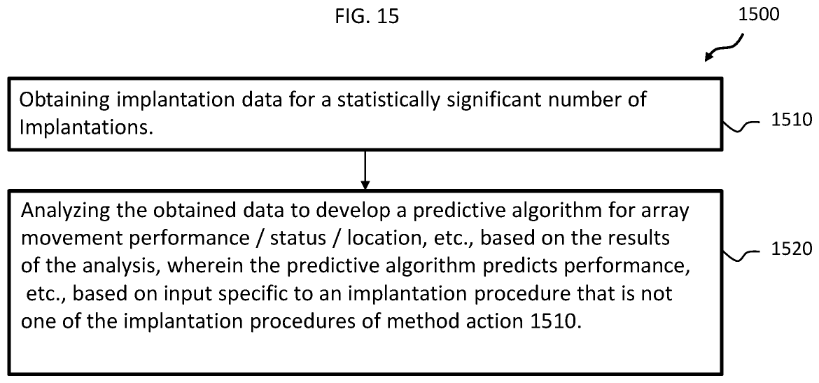

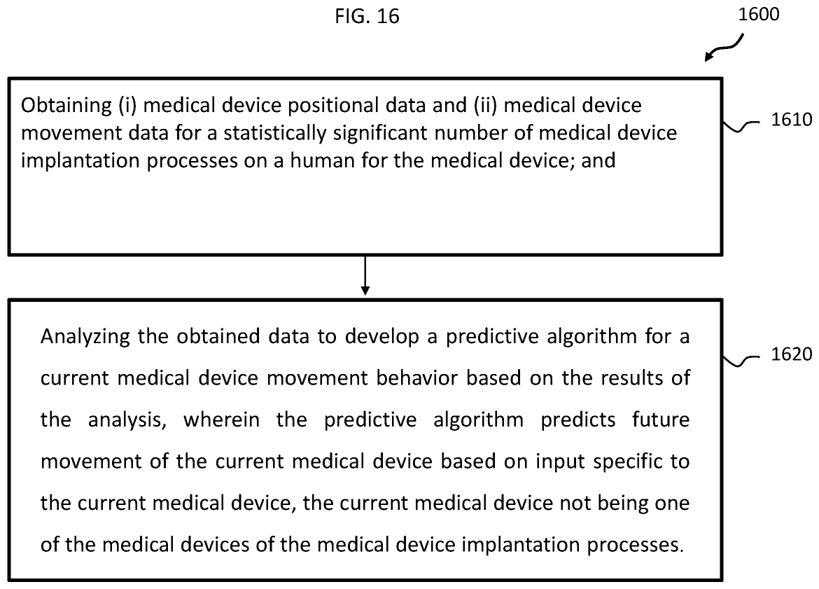

As an initial concept, briefly, FIG. 15 depicts an exemplary flowchart for an exemplary method, method 1500, of utilizing a model based on results from and/or that is a product of artificial intelligence, such as, for example, machine learning, such as a DNN, according to an exemplary embodiment. Method 1500 includes method action 1510, which includes obtaining data relating to actions taken during a plurality of medical device implantation procedures of a given kind (e.g., cochlear implant electrode array, pacemaker electrodes, retinal electrode arrays, etc.), results that were achieved during and/or as a result of a medical device implantation procedure, and/or occurrences related to that medical device implantation procedure. This data is obtained for a statistically significant number of sensory impaired individuals. FIG. 16 presents a flowchart for another method, method 1600, that includes method action 1610, which includes obtaining (i) medical device positional data and (ii) medical device movement data for a statistically significant number of medical device implantation processes on a human for the medical device.

In an exemplary embodiment, the obtained data includes, by way of example and not by way of limitation, with respect to, also by way of example and not by way of limitation, a cochlear implant electrode array:
speed and/or rate and/or acceleration, instantaneously and/or time averaged (distance averaged) over the entire process or in a segmented manner, such as the first third, the second third and the third third of the length of the array to be inserted or over a portion thereof, such as, for example, segments of less than, greater than and/or equal to 0.1, 0.2, 0.3, 0.4, 0.5, 0.6, 0.7, 0.8, 0.9 1, 2, 3, 4, 5, 6, 7, 8, 9, 10, 11, 12, 13, 14, 15, 16, 17, 18, 19, 20, 21, 22, 23, 24, 25, 26, 27,2 8, 29, 30, 31, 32, 33, 34, 35, 36, 37, 38, 39, 40, 41, 42, 43, 44, 45, 46 47, 48, 49, or 50%, or any value or range of values therebetween in 0.1 increment of the length of the array ultimately inserted into the cavity of the cochlea (not including the passage thereto) and/or the time to insert from first inserting into the passage to locating the array at is final location (where it is held for packing), and/or with respect to the portion thereof, such as ABC time and/or distance, where in an embodiment, the values can be discretized in accordance with the teachings above,
force on the electrode array during the insertion process, again instantaneously and/or time averaged etc., in a manner consistent with that just detailed with respect to the speed etc. ;
twist of the electrode array during the process, again in manners consistent with that just detailed;
distance from the modiolus wall, discretized and/or mean, mean, median and/or mode, instantaneously and/or time averaged etc., in a manner consistent with that just detailed with respect to the speed;
radius of curvature of the electrode array, discretized and/or averaged (mean, median and/or mode) with and/or without the above parsing;
the pose of the electrode array, with and/or without the above parsing;
depth of insertion, without and/or without the above parsing;
the movements of the array with and/or without the above parsing;
the status of the array, parsed as above;
piercing/puncturing scenarios, and/or depth thereof, with and/or without the above parsing;
demographic data, such as age, sex, genetic features, size of head, features associated with the cochlea (size, surface texture/roughness, surface features, which could be obtained by imaging (non-invasive or invasive), length of hearing loss, how hearing loss occurred, etc.
angle of insertion, with and/or without the above parsing;

Note that the above are not mutually exclusive (e.g., status and depth).

Note that in some embodiments, all of the above can be correlated to time and/or insertion depth.

Additional features can be used, such as, for example, history from a prior insertion in the same ear could be used. This could be a backup device after a failed first insertion, or in a revision surgery after device failure, the system could look at data from the original insertion. Another possibility is to look at data from implantation in the opposite ear. That is, the data from the left ear could be used by the system to forecast/predict/made various determinations during insertion about the right ear, and visa-versa. Embodiments thus include comparing features of the ears . of a given person and determining that the features are sufficiently comparable enough so that the data associated with one ear can be utilized during the insertion process for the other ear or that they are not sufficiently comparable enough so that the data cannot be used. In an exemplary embodiment, this could be based on preoperative imaging of both sides of the head of the recipient by way of example.

The specific anatomy of the patient based on pre-operative imaging could be included in the data analyzed by the systems and methods herein. By taking a large dataset of the core aspects of the anatomy (e.g., cochlea morphology, position of the facial nerve and/or chorda tympani) and records of the electrical measures of electrode position in the cochlea during insertion (e.g., angular depth and modiolar proximity), a training dataset could be developed for a machine-learning algorithm. This would enable forecasting of the maneuvers that are utilitarian for a future case which may provide a greater confidence in the selected maneuvers.

Note that if the feature is measured/analyzed/taken into account during the methods detailed herein it can be data that is collected to train the neural network. Corollary to this is that any of the above can be the input/data obtained by any of the systems and/or methods detailed herein for the evaluations/predictions.

And briefly, in an embodiment of method 1300 detailed above, that method further comprises receiving input based on a shape and/or a surface feature of a cochlea of the human, wherein the action of automatically analyzing includes automatically analyzing data based on the input based on shape and/or surface, to develop the recommended action. In an exemplary embodiment, a rough surface of the duct of the cochlea into which the electrode array is to be inserted is more conducive or otherwise more likely to create a situation where the tip is hung up within the cochlea, and thus there could be a tenfold scenario and/or a bowing of the array. In this regard, in an exemplary embodiment, the methods and/or algorithms and/or models and/or evaluations can take into account this increased likelihood and thus adjust the predictions and/or determinations and/or develop the recommendations, etc., accordingly.

In a similar vein, with respect to method 1000, that method can also include the action of providing demographic and/or physiological information about the human to the computer system, wherein the evaluation has also taken into account the provided demographic and/or physiological information.

Method action 1510 can be executed by obtaining and otherwise documenting insertion processes of cochlear implants for a number of implantation procedures, which can be done manually and/or electronically, such as for example with the use of imaging devices to determine various features of the electrode array, or with respect to the utilization of the insertion devices detailed herein, or other types of sensors. Any device, system and/or method that can enable the acquisition of data that can have utilitarian value, such as the data detailed above, can be utilized in at least some exemplary embodiments providing that the art enables such.

It is also noted that in at least some exemplary embodiments, method action 1510 can be executed such that the person who is the subject of the method action is at a remote location from the entity obtaining the data/the entity executing method action 1510.

In an embodiment, there can be statistical models and/or probabilistic models/algorithms that can be used to implement some teachings herein. In an embodiment, these models and/or machine learning models, etc., are developed by analyzing utilitarian amounts of historically collected treatment data (e.g., preoperative and postoperative data of cochlear implant patients). In an embodiment, depending on the data collected and/or the nuances of the population associated with the data that is collected (if the population is statistically similar in a given category, less data will be needed than if the population has a high number of people who are statistically different in a given category) the amounts of collected data (number of procedures) can correspond to data for less than greater than or equal to 50, 60, 70, 80, 90, 100, 125, 150, 175, 200, 250, 300, 350, 400, 500, 600, 700, 800, 900, 1000, 1250, 1500, 1750, 2000, 3000, 4000, 5000, 6000, 7000, 8000, 9000, or 10000 implantations, or any value or range of values therebetween in 1 increment (e.g., 73, 555, 321 to 4,444 people, etc.).

The data analyzing algorithms to develop the model(s) can include by way of example:
(i) curation of the raw dataset/raw historical data to remove and/or adjust data of insertions/implantations where records are sparely populated or are of low quality;
(ii) imputation of the dataset/raw historical data to fill in missing data using patterns and relationships within the dataset;
(iii) transformation of information into numerical forms;
(iv) transformation of preoperative factors in principle components (using principal component analysis) or independent components (independent component analysis);
(v) identification of preoperative factors and/or transformed factors that correlate and/or causally relate to the outcomes metrics; and/or
(vi) machine learning training (or AI training), which can include calculation of coefficients, parameters, thresholds and/or weights of models by minimizing a cost function such as minimizing the sum of squared error.

As noted above, in some embodiments, method action 1510 includes obtaining data that is tailored to specific subsets of the human, such as data relating to at least demographic data and sensory performance of the human.

Method 1500 further includes method action 1520, which includes analyzing the obtained data to develop a predictive algorithm for array movement performance/status/location, etc., based on the results of the analysis, wherein the predictive algorithm predicts performance, etc., based on input specific to an implantation procedure that is not one of the implantation procedures of method action 1510. Again, in an embodiment, the action of analyzing the obtained data is executed using machine learning. Method 1600 includes method action 1620, which includes analyzing the obtained data to develop a predictive algorithm for a current medical device movement behavior based on the results of the analysis, wherein the predictive algorithm predicts future movement of the current medical device based on input specific to the current medical device, the current medical device not being one of the medical devices of the medical device implantation processes.

In an embodiment, the action of analyzing the obtained data is executed using machine learning. The use of machine learning can include calculating coefficients, parameters, thresholds and/or weights of models by minimizing a cost function. The action of analyzing the obtained data machine-trains a system that results in the developed predictive algorithm. In an embodiment, method 1500 and/or 1600 includes the action of developing a cohort model based on the obtained data, wherein the action of analyzing the obtained data results in the development of a cohort model based on the obtained data, and the predictive algorithm uses the cohort model in a brute force manner to predict future movement of the current medical device based on input specific to the current medical device. In an embodiment, of the obtained data, at least a portion thereof is used, in a neural network, for training and at least a portion thereof is used, in the neural network, for verification. In an embodiment, the machine learning develops the predictive algorithm by internally identifying important features from the obtained data. In an embodiment, the method includes curation of the obtained data to remove data having records that are sparely populated and/or are low quality and/or filling in missing data using patterns and/or relationships within the obtained data to obtain filled in data, which filled in data includes the obtained data and/or analyzing the filled in data to develop the predictive algorithm, wherein the action of analyzing the filled in data includes the action of analyzing the obtained data.

The predictive algorithm that predicts performance based on input specific to an implantation that is not one of the implantations of the statistically significant dataset can be what is used to execute the methods above. The action of analyzing the obtained data can be executed using a machine network, neural network, DNN, to develop the predictive algorithm. Method action 1520 is executed utilizing, for example, the DNN detailed above, although in other embodiments, any other type of machine learning algorithm or AI algorithm can be utilized in at least some exemplary embodiments. Consistent with the teachings further below, in an exemplary embodiment, the predictive algorithm is not focused on a specific feature. Instead, it utilizes a plurality of features that are unknown or otherwise generally represent a complex arrangement (if such can even be considered features in the traditional sense).

Still further, in some exemplary embodiments of method 1500, consistent with the teachings detailed herein, the action of analyzing the obtained data machine-trains a system that results in the developed predictive algorithm. Also, still with respect to method 1500, in an exemplary embodiment, of the obtained, at least a portion thereof is used, in the neural network, for training and at least portion thereof is used, in the neural network, for verification. Also, in this exemplary embodiment, the neural network develops the predictive algorithm by or transparently (sometimes, invisibly) identifying important features from the obtained data. Also consistent with the teachings herein, the predictive algorithm utilizes an unknowable number of features present in the speech data to predict hearing loss.

Concomitant with the teachings above, in an embodiment, the action of analyzing the obtained data machine-trains a system that results in the developed predictive algorithm. As noted above, in some embodiments, the use of machine learning includes calculating coefficients, parameters, thresholds and/or weights of models by minimizing a cost function.

In some embodiments, at least a portion of the obtained data is used, in a neural network (or whatever AI system is used), for training and at least a portion thereof is used, in the neural network, for verification. Also, in this exemplary embodiment, the neural network develops the predictive algorithm by transparently (sometimes, invisibly) identifying important features from the obtained data. Also consistent with the teachings above, the predictive algorithm utilizes an unknowable number of features present in the speech data to predict the output.

With respect to the unknown/transparent features, these can be features that are not related to a simple data description.

In an exemplary embodiment, the machine learning develops the predictive algorithm by internally identifying important features from the obtained data. For example, in implementing method 1500, action 1520 is executed utilizing, for example, the DNN detailed above, although in other embodiments, any other type of machine learning algorithm can be utilized in at least some exemplary embodiments. Consistent with the teachings detailed above, in an exemplary embodiment, the predictive algorithm is not focused on specific feature. Instead, it utilizes a plurality of features that are unknown or otherwise generally represent a complex arrangement (if such can even be considered features in the traditional sense). Thus, the predictive algorithm utilizes a complex arrangement number of features present in the data obtained in method action 1510 to predict the performance and/or to determine the actions to be taken.

As noted above, exemplary embodiments include utilizing a trained neural network (or whatever artificial intelligence system is applicable) to implement or otherwise execute at least one or more of the method actions and/or functionalities detailed herein, and thus embodiments include a trained neural network configured to do so. Exemplary embodiments also utilize the knowledge of a trained neural network/the information obtained from the implementation of a trained neural network to implement or otherwise execute at least one or more of the method actions/functionalities detailed herein, and accordingly, embodiments include devices, systems, and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network. The teachings detailed herein include utilizing machine learning methodologies and the like to establish systems and devices and computers to implement the teachings herein.

In an exemplary embodiment, the actions of evaluating/analyzing, etc., can be executed on raw data and/or modified data or filtered data or transformed data (hence analyzing data based on the obtained data—this can be the data or data that has been transformed). In an exemplary embodiment, the product of and/or the results of machine learning, or otherwise artificial intelligence, used to execute the analysis/evaluation, etc., and to otherwise make the predictions/determinations, etc., is a chip that is fabricated based on the results of machine learning. In an exemplary embodiment, the product/result is a neural network, such as a deep neural network (DNN). The product can be based on or be from a neural network. In an exemplary embodiment, the product/results is code. In an exemplary embodiment, the product/results is a logic circuit that is fabricated based on the results of machine learning. The product/results can be an ASIC (e.g., an artificial intelligence ASIC). The product/results can be implemented directly on a silicon structure or the like. Any device, system, and/or method that can enable the results of artificial intelligence to be utilized in accordance with the teachings detailed herein, such as in a hearing prosthesis or a component that is in communication with a hearing prosthesis, can be utilized in at least some exemplary embodiments. Indeed, as will be detailed below, in at least some exemplary embodiments, the teachings detailed herein utilize knowledge/information from an artificial intelligence system or otherwise from a machine learning system.

In an embodiment, the systems and devices and/or methods herein use prediction and/o or determination models to execute one or more or all of the “analytical” actions/functionalities detailed herein, and the models can be results of machine learning/a product of machine learning (either directly or indirectly/based thereon), or a DNN, etc., that has the functionality thereof, and executes the functionality based on input thereto in view of its training. Again, the prediction model/determination model can be/or can be part of/can be established/non-transitorally based in computer chip (as opposed to a processor) or an electronic circuit. The models can be electronics that have the function thereof. Consistent with the well-known phenomenon of machine learning, it may not be that the prediction and/or determination model can be specifically understood. It may not be known exactly how the prediction model works, consistent with how artificial intelligence works in general and machine learning works specifically. The prediction model can be the results of the machine learning as noted above. This can be the machine learning as frozen in time (when the learning was halted to establish the product). In an exemplary embodiment, an algorithm can include and/or the models herein can be based on a linear model, such as y=β₀+Σβᵢxᵢ+ε. Here, y is the outcome being predicted, β are coefficients and x are inputs.

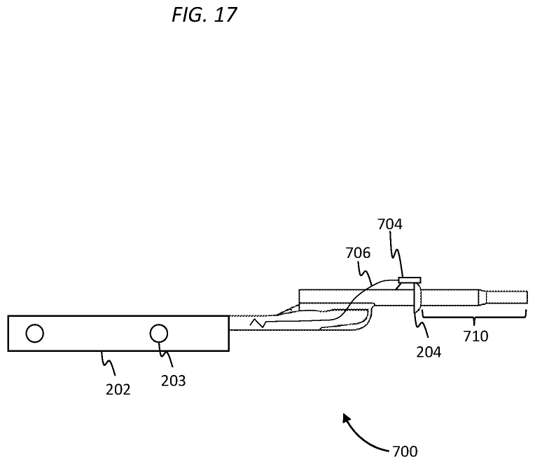

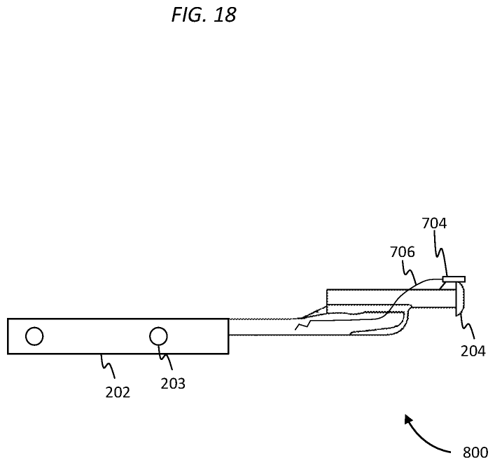

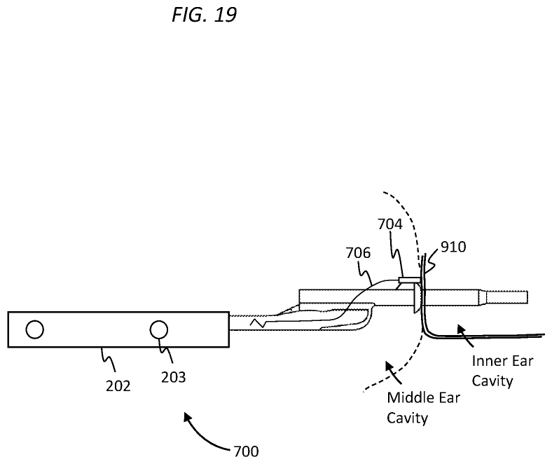

FIG. 17 depicts an exemplary embodiment of a cochlear electrode array insertion guide 700. In an exemplary embodiment, the insertion guide 700 corresponds to that of the insertion guide 200 detailed above, with the exception of the addition of electrode 704, and the modifications to the tool so as to support the electrode and the associated components thereof (e.g., electrical leads 706 (only the “distal” portion of the lead (distal relative to the tool 800) is depicted, the “break’ being conceptual), etc.—more on this below). Accordingly, FIG. 17 depicts a cochlear electrode array insertion guide comprising an array guide (e.g., the insertion guide tube (210 of FIG. 2)) and an active functional component (e.g., electrode 704). Some additional details of some exemplary functional components, including some exemplary active functional components, will be described in greater detail below. However, it is briefly noted at this time that not all embodiments of the cochlear electrode array insertion guide include an intracochlear portion. In this regard, FIG. 17 depicts a tool 700 that includes an intracochlear portion 710. This is the portion to the right of stop 204/the portion on the distal side of stop 204 (distal relative to the entire insertion guide). Conversely, FIG. 18 depicts a tool 800 that does not include an intracochlear portion. Instead, stop 204 is configured to be placed against the outside of the cochlea such that the passageway through the tool through which the electrode array is passed is aligned with the pertinent window and/or cochleostomy such that no parts of the tool 800 enters the cochlea.

It is noted that while the teachings detailed herein with respect to extra functionality of the insertion guide are based on the insertion guide detailed above with respect to FIGS. 5A-6B, these teachings can be applicable to other types of insertion guides. Indeed, as will be detailed below, some embodiments of the insertion guides do not have an intracochlear portion at all. Accordingly, the teachings above with respect to FIGS. 5A-6B serve as but one example of an insertion guide that the following teachings can be utilized in conjunction therewith.

With reference back to FIG. 17, the exemplary active functional component can be an electrode (read or energizing, etc.).

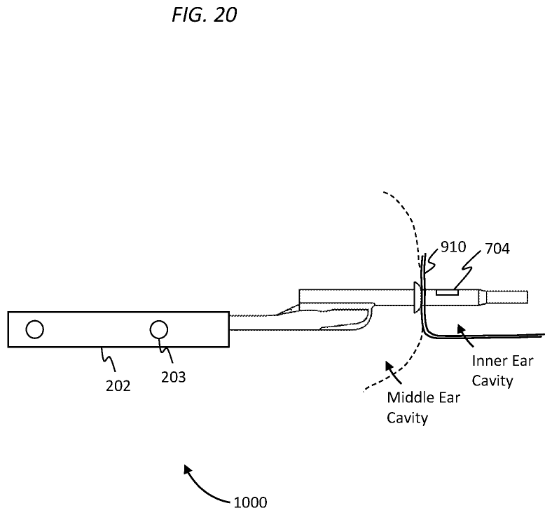

While the embodiments detailed above have focused on the electrode being located entirely outside the cochlea (e.g., entirely inside the middle ear), in an alternative embodiment, the electrode is located inside the cochlea during use. FIG. 20 depicts an exemplary insertion regime utilizing exemplary electrode array insertion guide 1000 where the electrode is located entirely in the inner cavity (in the cochlea) when the insertion guide is fully inserted into the inner ear cavity. Still further, FIG. 21 depicts an exemplary insertion regime utilizing exemplary electrode array insertion guide 1100 where the electrode being is located in the wall that separates the middle ear cavity from the inner ear cavity when the insertion guide is fully inserted into the inner ear cavity.

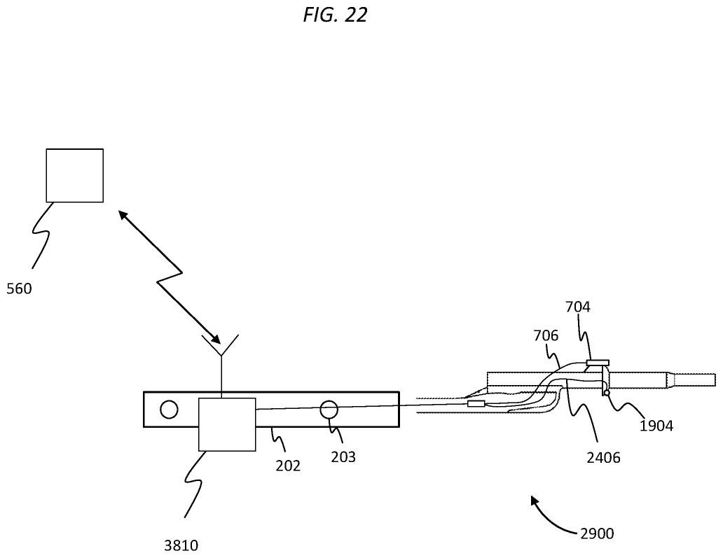

FIG. 22 depicts an insertion guide 2900 that is in wireless communication via element 3810 with a remote component 560, which could be a test unit or a control unit as disclosed further below.

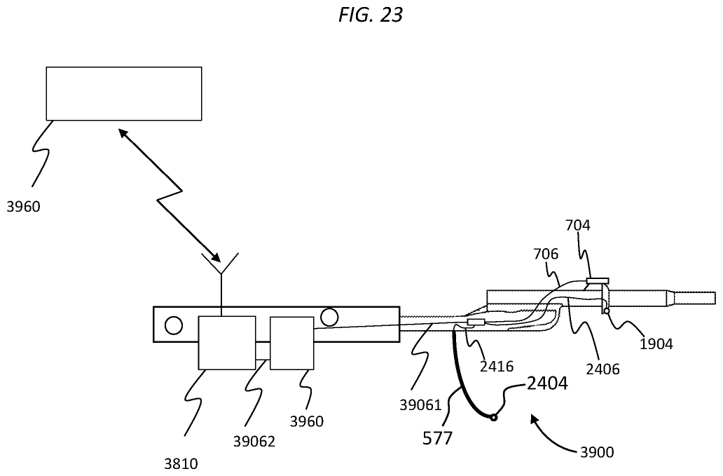

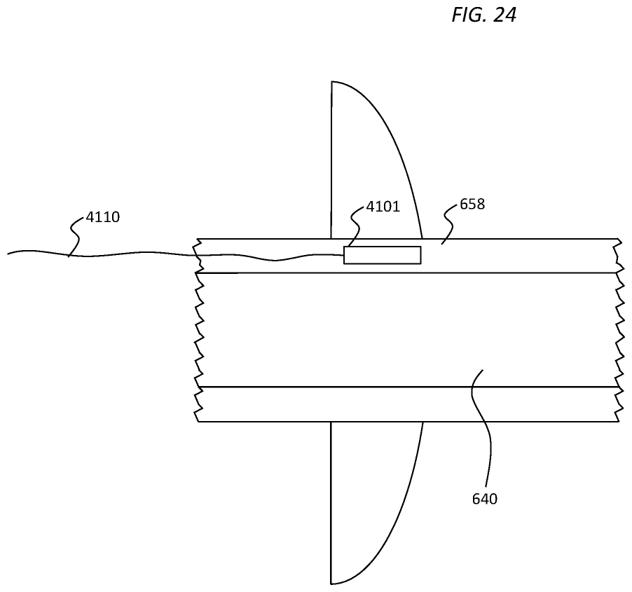

As briefly noted above, in at least some exemplary embodiments, some exemplary insertion guides can include a self-contained measurement system. FIG. 23 depicts such an exemplary embodiment of an insertion guide 3900. Insertion guide 3900 contains a complete measurement system. As can be seen, the insertion guide 3900 further includes a reference electrode 2404, which is in signal communication with the electrical leads of the system via lead 2416. Lead 39061 extends from the connector to test unit 3960, which can correspond to test a test unit configured to executes one or more or all of the test teachings herein, and can be a personal computer programmed to execute such. Test unit 3960 is in signal communication with communication unit 3810 via lead 39062. Communications unit 3810 can be in wireless communications with remote device 3960. In an exemplary embodiment, the remote device 3960 is a data storage device/data recording device that records the data transmitted via the communications unit 3810. For example, 3960 can be a desktop and/or a laptop computer having memory therein to record the data. In an alternate embodiment, device 3960 can be a control unit or the like, again such as a computer, that can control measurement system of the guide 3900. That said, in an exemplary embodiment, the guide 3900 includes an activation switch or the like so that the system can be activated and/or deactivated by the surgeon or other healthcare professional.

FIG. 24 depicts another exemplary embodiment of an insertion guide that has a functionality beyond that of an electrode array support/an electrode array insertion device. Particularly, the embodiment of FIG. 24 depicts a portion of the insertion guide tube at the stop 204 where a sensor 4101 is located in the wall 658 of the tube, although in other embodiments, the sensor 4101 is located on the inside wall of the tube and in other embodiments, the sensor 4101 is located on the outside wall of the tube. In this exemplary embodiment, the sensor is configured to sense or otherwise detect individual electrodes in the array as they pass by the sensor as the electrode array is inserted through the lumen 640 into the cochlea, and output a signal via lead 1410 indicative of at least one of an electrode passing the sensor 4101 or, in a more sophisticated embodiment, the speed of the electrode/electrode array passing by sensor 4101. In an exemplary embodiment, the sensor 4101 can be a sensor that utilizes capacitive sensing. In an exemplary embodiment, it could be a Hall effect sensor. In some embodiments, the sensor could be a sensor that comes into direct contact with the electrodes of the electrode array. In an exemplary embodiment, there is a system that receives the signal from lead 1410 and outputs data indicative of the insertion speed of the electrode. In an exemplary embodiment, the system can be a personal computer with an algorithm that analyzes the signal 4110, and outputs data to the surgeon. Exemplary output can be output by a speaker or the like indicating the speed of the insertion of the electrode array. (Feedback can be provided to a surgeon conducting a manual insertion such as by an audio system, or a visual system, the latter of which could be provided in a heads-up type display within the visual field of an operating microscope.)

Exemplary output can be output by a visual device indicating the speed of insertion of the electrode array. Exemplary output can correspond to the speed of insertion, a go/no go data package (e.g., insertion too fast/insertion speed fine). Such can be done via audio and/or visual devices. For example, a green light can indicate acceptable speed and a red light can indicate an unacceptable speed. Moreover, the system can be binary. The activation of the light will indicate that the speed is too fast/the audio indication (which could be a buzzer or a tone, etc.) activates when the insertion speed is too fast. The alternative could also be the case. The tone and/or light can be activated while the insertion speed is acceptable, and the tone or light is deactivated when the insertion speed is unacceptable. It will be noted that these indicators can also be utilized to indicate other sensed phenomenon or otherwise detected phenomenon as detailed herein.

In an exemplary embodiment, any of the teachings of US Patent Application Publication No. 2018/0050196, to inventor Nicolas Pawsey, Published on Feb. 22, 2018, can be used to insert the array, and the teachings therein can be combined with the present teachings to implement the teachings herein.

Any arrangement of the insertion guide that can enable the teachings herein can be used in some embodiments.

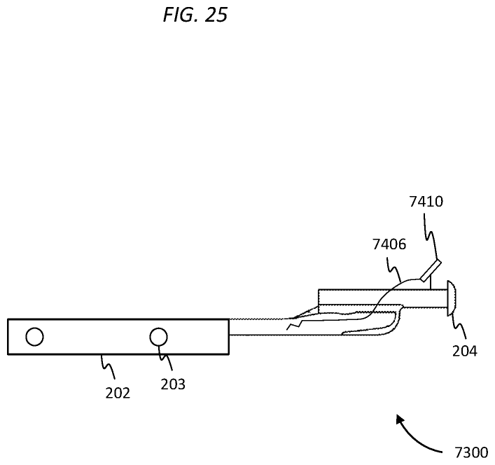

As noted above, the insertion guide can incorporate visual indicators to provide intraoperative feedback to the surgeon. As detailed above, exemplary embodiments have LEDs or the like arrayed about the stop. Still further, in an exemplary embodiment, a liquid crystal display or the like can be incorporated in or on the insertion guide. In this regard, FIG. 25 depicts an exemplary embodiment of an insertion guide 7300 which includes LCD 7410 mounted on the insertion guide tube. LCD 7410 is in electrical communication with other components of the guide and/or other systems remote from the guide via electrical lead 7406. In an exemplary embodiment, the LCD can provide text and/or numerical data to the surgeon during implantation/insertion of the electrode array. This can provide the instruction/recommendation as noted above to the surgeon. The LCD or the other visual indicators can be located anywhere on the guide that will be within the surgeon's immediate field-of-view, but also where the indicator will not obstruct the surgeon's field-of-view of the pertinent portions of the anatomy of the recipient and/or the pertinent portions of the guide 7300 during insertion of the electrode array. In an exemplary embodiment, the indicators provide information pertaining to insertion depth, which can include the absolute depth and/or an indication that the electrode array has reached the intended or programmed stopped depth. Indication can be an insertion speed, which can be absolute speed of insertion or can be an indication that the insertion speed limit has been exceeded. The indication can be an adverse measurement indication. This measurement can be a general indication, such as an indicator that something has gone wrong whatever that is, or specific indication, such as an indication explicitly relating to tip fold over, basilar membrane contact, scala dislocation, etc. Accordingly, in an exemplary embodiment, such indication can correspond to any of the anomalous electrode position indicators detailed herein.

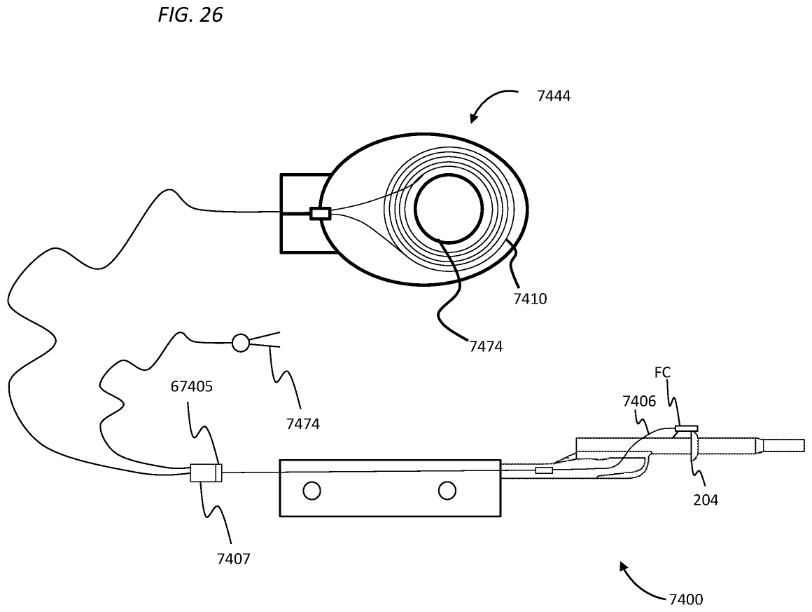

As noted above, embodiments include an insertion guide configured to communicate with a receiver/stimulator of a cochlear implant. In this regard, FIG. 26 depicts an exemplary insertion guide 7400 which is presented by way of concept. Insertion guide 7400 is a functional component FC mounted thereon. This functional component is representative of any of the additional functionalities of the insertion guide detailed herein and/or variations thereof. For example, element FC could be an electrode, it could be the acoustic stimulation generator, or it could be the ultrasonic transducer. FC could also be any of the indicators detailed herein (e.g., the LCD screen). As can be seen, insertion guide 7400 includes connector 64705 in electrical communication with the functional component FC via electrical lead 746. Connector 64705 is connected to connector 7407 of inductance coil 7444. In an exemplary embodiment, inductance coil 7444 includes coil 7410 configured to establish a magnetic inductance field so as to communicate with the corresponding coil of the receiver-stimulator of the cochlear implant. Inductance coil 7444 includes a magnet 7474 so as to hold the inductance coil 7474 against the coil of the receiver/stimulator of the cochlear implant in a manner analogous to how the external component of the cochlear implant is held against the implanted component, and how the coils of those respective components are aligned with one another. While the embodiment depicted in FIG. 26 depicts no other functional component between the functional component FC and the inductance coil 7444, in an alternate embodiment, one or more of the units detailed herein can be located there between. By way of example, generator 6520 with respect to the insertion guide 6500 detailed above can be located therebetween or otherwise be in signal communication with the leads so as to establish communication with that element with the cochlear implant. In an exemplary embodiment, a communications unit or the like is located between or otherwise is in signal communication with the leads so as to establish communication with the cochlear implant receiver-stimulator. In an exemplary embodiment, the insertion guide includes logic or a processor or other type of control unit that enables the insertion guide to work in conjunction with the cochlear implant so as to execute any of the methods detailed herein, such as, for example, where one or more electrodes of the electrode array insertion guide are utilized in a state of one or more electrodes of the electrode array as taught in those applications.

FIG. 26 also shows second lead from connector 7407 extending to alligator clip 7474, which in an exemplary embodiment, configured to clip onto the hard ball and/or the can of the implant, in which clip is in electrical communication with one or more electrodes on the electrode array that would be inside and/or outside of the cochlea during insertion. Indeed, it is also noted that in an exemplary embodiment, the entire portion that is inserted into the cochlea of the insertion guide can be the electrode, and thus be in electrical communication with the alligator clip 7474.

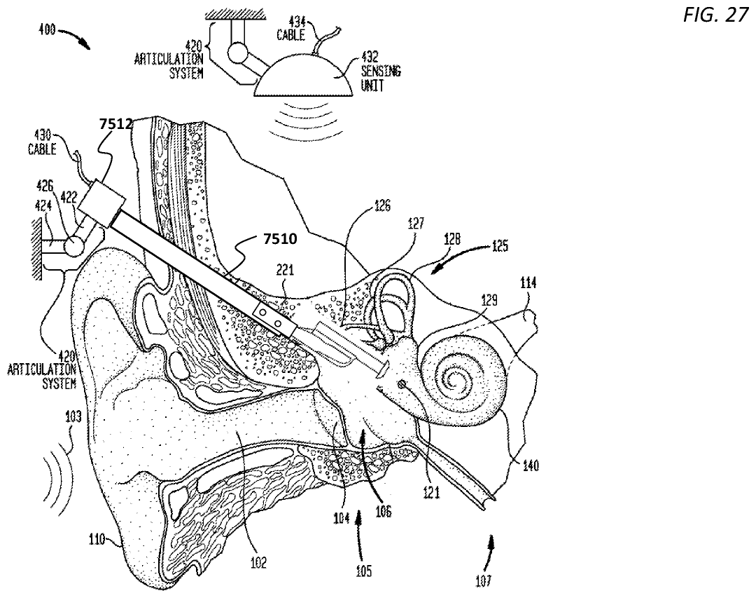

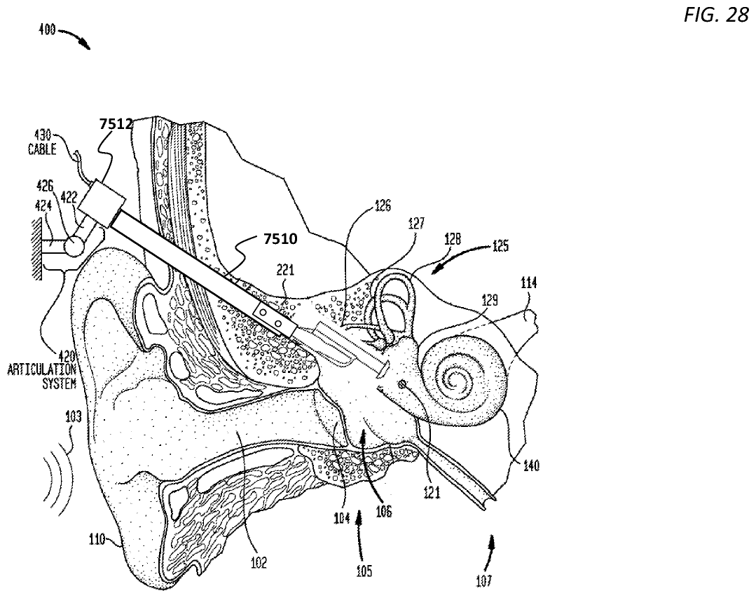

It is noted that at least some exemplary embodiments include utilization of the insertion guides detailed herein and/or variations thereof with a robotic electrode array insertion system. In this regard, FIG. 27 is a perspective view of an exemplary embodiment of an insertion system 400. It is noted that the embodiment depicted in FIG. 27 is presented for conceptual purposes only. Features are provided typically in the singular show as to demonstrate the concept associated therewith. However, it is noted that in some exemplary embodiments, some of these features are duplicated, triplicated, quadplicated, etc. so as to enable the teachings detailed herein and/or variations thereof. Briefly, it is noted that any teaching detailed herein can be combined with a robotic apparatus and/or a robotic system according to the teachings detailed herein and/or variations thereof. In this regard, any method action detailed herein corresponds to a disclosure of a method action executed by a robotic apparatus and/or utilizing a robot to execute that action and/or executing that method action is part of a method where other actions are executed by robot and/or a robotic system etc. Still further, it is noted that any apparatus detailed herein can be utilized in conjunction with a robotic apparatus and/or a robot and/or a system utilizing such. Accordingly, any disclosure herein of an apparatus corresponds to a disclosure of an apparatus that is part of a robotic apparatus and/or a robotic system etc. and/or a system that includes a robotic apparatus etc.

System 400 includes a robotic insertion apparatus including arm 7510 to which insertion guide 200 or any other insertion guide according to the teachings detailed herein and/or variations thereof is attached (e.g., bolted to arm 7510). In this exemplary embodiment, arm 7510 is depicted as a single structure extending from the insertion guide to mount 7512. However, in an alternate embodiment, arm 7510 can be a multifaceted component which is configured to articulate at various locations thereabout.

In an exemplary embodiment, arm 7510 is releasably connected by way of a releasable connection to mount 7512, which is supported by a support and movement system 420, comprising support arm 422 which is connected to joint 426 which in turn is connected to support arm 424. Support arm 424 is rigidly mounted to a wall, a floor, or some other relatively stationary surface. That said, in an alternative embodiment, support arm 424 is mounted to a frame that is attached to the head of the recipient or otherwise connected to the head of the recipient such that global movement of the head will result in no relative movement of the system 400 in general, and the insertion guide in particular, relative to the cochlea. Joint 426 permits arm 2510, and thus the insertion guide, to be moved in one, two, three, four, five, or six degrees of freedom. (It is noted again that FIG. 27 is but a conceptual FIG.—there can be joints located along the length of arm 7510, so as to enable arm 75102 articulate in the one or more of the aforementioned degrees of freedom at those locations. In an exemplary embodiment, joint 426 includes actuators that move mount 7512, and thus the insertion guide, in an automated manner, as will be described below. In an exemplary embodiment, the system is configured to be remotely controlled via communication with a remote control unit via communication lines of cable 430. In an exemplary embodiment, the system is configured to be automatically controlled via a control unit that is part of the system 400. Additional details of this will be described below.

The system 400 further includes by way of example only and not by way of limitation, sensor/sensing unit 432. That said, in some embodiments, sensor 432 is not part of system 400. In some embodiments, it is a separate system. Still further, in some embodiments, it is not utilized at all with system 400. While sensor 432 is depicted as being co-located simultaneously with the insertion guide, etc., as detailed below, sensor 432 may be used relatively much prior to use of the insertion guide. Sensing unit 432 is configured to scan the head of a recipient and obtain data indicative of spatial locations of internal organs (e.g., mastoid bone 221, middle ear cavity 423 and/or ossicles 106, etc.) In an exemplary embodiment, sensing unit 432 is a unit that is also configured to obtain data indicative of spatial locations of at least some components of the insertion guide and/or other components of the robotic apparatus attached thereto. The obtained data may be communicated to remote control unit 440 via communication lines of cable 434. As may be seen, sensor 432 is mounted to a support and movement system 420 that may be similar to or the same as that used by the robotic apparatus supporting the insertion guide.

In an exemplary embodiment, sensing unit 432 is an MRI system, an X-Ray system, an ultrasound system, a CAT scan system, or any other system which will permit the data indicative of the spatial locations to be determined as detailed herein and/or variations thereof. As will be described below, this data may be obtained prior to surgery and/or during surgery. It is noted that in some embodiments, at least some portions of the insertion guide are configured to be better imaged or otherwise detected by sensing unit 432. In an exemplary embodiment, the tip of the insertion guide includes radio-opaque contrast material. The stop of the insertion guide can also include such radio-opaque contrast material. In an exemplary embodiment, at least some portions of insertion guide in general, and the robotic system in particular, or at least the arm 7510, mount 7512, arm 422, etc., are made of non-ferromagnetic material or other materials that are more compatible with an MRI system or another sensing unit utilized with the embodiment of FIG. 27 than ferromagnetic material or the like. As will be described in greater detail below, the data obtained by sensing unit 432 is used to construct a 3D or 4D model of the recipient's head and/or specific organs of the recipient's head (e.g., temporal bone) and/or portions of the robotic apparatus of which the insertion guide is a part. That said, to be clear, in some embodiments, sensing unit 432 is not present, as seen in FIG. 28. Noter that these imaging/sensing systems can be used to determine one or more of the spatial features detailed herein.

It is also noted that in some exemplary embodiments of system 400, there are actuators or the like that drive the electrode array through the insertion guide into the cochlea. These actuators can be in signal communication with the control unit. In an exemplary embodiment, the control unit can control the actuators to push the electrode array into and/or out of the cochlea as will be described in greater detail below. Concomitant with the robotic assembly supporting the insertion guide, in an exemplary embodiment, the control unit is configured to automatically control these actuators.

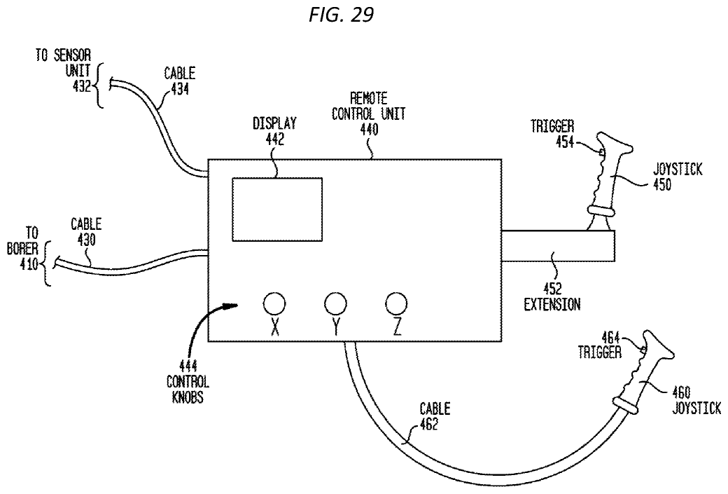

FIG. 29 is a simplified block diagram of an exemplary embodiment of a remote control unit 440 for controlling the robotic apparatus supporting the insertion guide and sensing unit 432 via communication lines 430 and 434, respectively. Again, it is noted that in some alternate embodiments, the remote control unit 440 is an entirely automated unit. That said, in some alternate embodiments, the remote control unit can be operated automatically as well as manually, which details will be described below.

Remote control unit 440 includes a display 442 that displays a virtual image of the mastoid bone obtained from sensor 432 and may superimpose a virtual image of the insertion apparatus onto the virtual image indicative of a current position of the drill bit relative to the ear anatomy. An operator (e.g., surgeon, certified healthcare provider, etc.) utilizes remote control unit 440 to control some or all aspects of the robotic apparatus and/or sensing unit 432. Exemplary control may include depth of insertion guide insertion, angle of guide insertion, speed of advancement and/or retraction of electrode array, etc. Such control may be exercised via joystick 450 mounted on extension 452 which fixedly mounts joystick 450 to a control unit housing. Such control may be further exercised via joystick 460 which is not rigidly connected to housing of remote control unit 440. Instead, it is freely movable relative thereto and is in communication with the remote control unit via communication lines of cable 462. Joystick 462 may be part of a virtual system in which the remote control unit 440 extrapolates control commands based on how the joystick 462 is moved in space, or joystick may be a device that permits the operator more limited control over the cavity borer 410. Such control may include, for example an emergency stop upon release of trigger 464 and/or directing the robot to drive the insertion guide further into the cochlea by squeezing the trigger 464 (which, in some embodiments, may control a speed at which the insertion guide is advanced by squeezing harder and/or more on the trigger). In the same vein, trigger 454 of joystick 450 may have similar and/or the same functionality.

Control of the robot assembly supporting the insertion guide may also be exercised via knobs 440 which may be used to adjust an angle of the insertion guide in the X, Y and Z axis, respectively. Other controls components may be included in remote control 440.

FIG. 29 depicts an exemplary insertion guide which can correspond to any of the insertion guide detailed herein and/or variations thereof, or any other insertion guide for that matter, further including an electrode array insertion actuator 7720. In an exemplary embodiment, actuator assembly 7720 includes a passageway therethrough through which the electrode array extends. The actuator assembly drives the electrode array in a manner replicating that by which the surgeon pushes the electrode array forward along the insertion guide and into the insertion tube and thus into the cochlea.

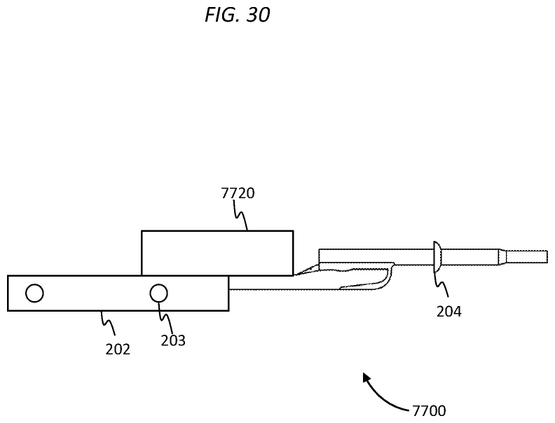

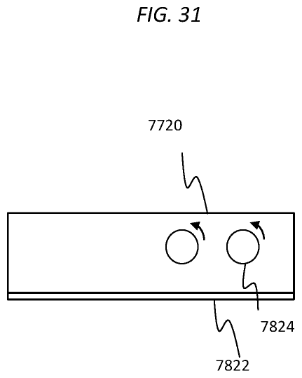

FIG. 30 depicts an exemplary embodiment of the actuator assembly 7720. As can be seen, actuator assembly includes two actuators 7824 in the form of wheels mounted to electric motors that rotate the wheels in a counterclockwise direction so as to advance the electrode array, and in a clockwise direction so as to retract the electrode array. Actuator assembly 7720 further includes a floor 7822. The floor 7822 works in combination with the actuators 7824 so as to “trap” the electrode array there between with a sufficiently compressive force so that the friction forces between the actuators 7824 and the electrode array enable the actuators 7824 to drive the electrode array forward and/or backwards, but not enough so as to damage the electrode array. FIG. 31 depicts an exemplary movement of the wheels 7824.

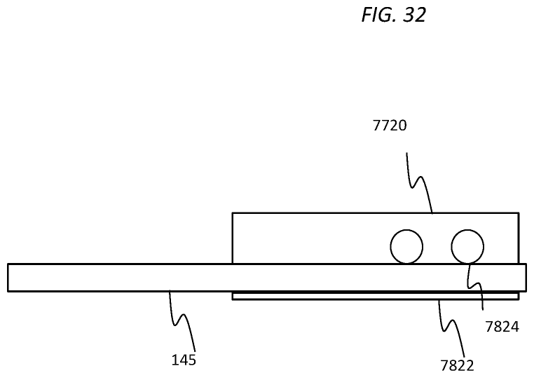

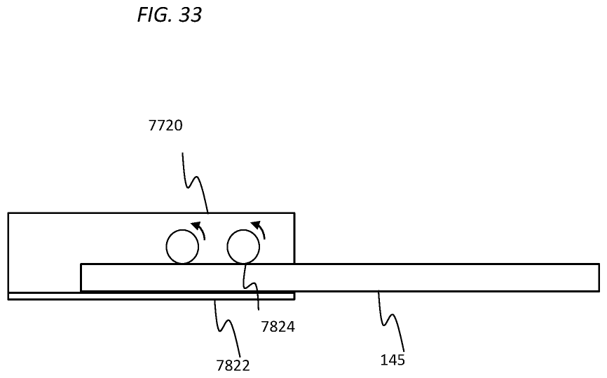

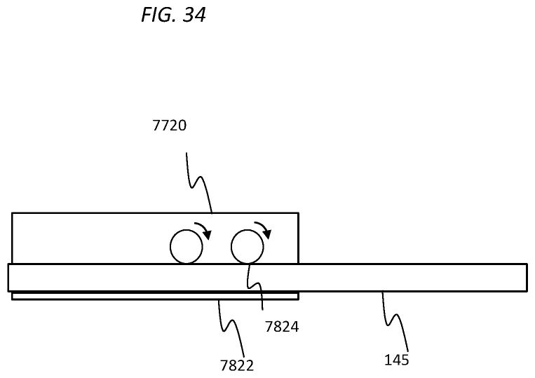

FIG. 32 functionally depicts an electrode array 145 “loaded” in actuator assembly 7720 prior to driving the electrode array into the insertion sheath. FIG. 33 functionally depicts the electrode array being driven forward (FIG. 33 is depicted in a functional manner—in reality, the electrode array 145 would extend up the ramp and then into the insertion sheath), and FIG. 34 functionally depicts the electrode array being retracted from the position seen in FIG. 33.

While the embodiment of the actuator assembly depicted in FIG. 32 includes two top actuators, in an alternate embodiment, only one top actuator is utilized and/or in another embodiment, three or four or five or six or more actuators are utilized. Also, in an exemplary embodiment, one or more bottom actuators can also be utilized. Note also that instead of the actuators being located on the top and the floor 7822 being on the bottom, the actuators can be located on the bottom and the floor can be located on the top.

It is noted that while the embodiment of FIG. 32 is depicted utilizing actuators having round wheels, in an alternate embodiment, other types of working and of the actuators can be utilized.

To be clear, the embodiment of FIG. 29 depicted above can also include the actuator assembly's detailed herein and/or variations thereof. That is, insertion guide 7700 can be attached to the arm 7510 of the system 400. Moreover, the actuators of the actuator assembly can be placed into signal communication with the control unit 440 or any other control unit of the system 400 to enable the control unit to advance and/or retract the electrode array. Note also that in some alternate embodiments, the system 400 is such that the only non-manually actuating component is the actuator assembly. That is, in an exemplary embodiment, system 400 can be such that the frame of the like is placed around the recipient's head and secured thereto, and the arm 7510 supporting the insertion guide attached thereto can be moved manually by the surgeon, such that the surgeon can align or otherwise place the insertion guide into the cochlea. In this regard, by way of example only and not by way of limitation, the insertion guide can be configured so as to attached to the arm 7510 on a trolley or the like. In an exemplary embodiment, the surgeon moves arm 7510 into position so that the insertion guide is aligned with the cochlea, at the desired angle, etc., and then be surgeon manually pushes the insertion guide forward into the cochlea (in the case of an intra-cochlear insertion guide) or against the cochlea in the case of a non-intra-cochlea insertion guide). After that, the actuator assembly can be utilized in a remote-controlled and/or automated manner.

That said, in an alternate embodiment, the general positions of the system 400 can be established utilizing manual methods, and then the positions can be refined utilizing automated/remote controlled methods (e.g., the actuators on the arm 7510 and/or the actuator at joint 426 can be actuated so as to finally position the insertion guide.

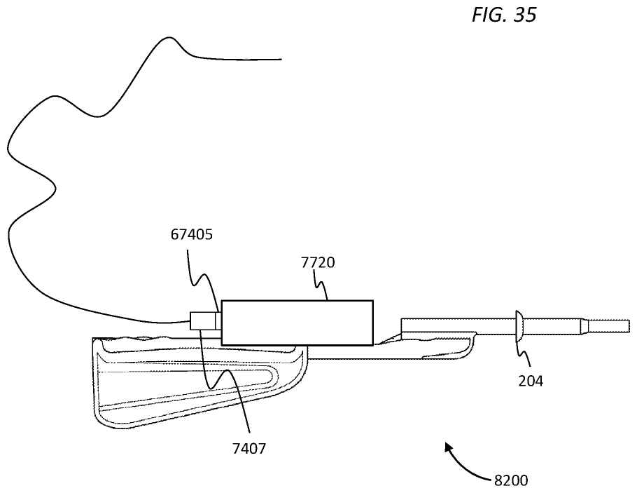

Note also that in some exemplary embodiments, the actuator assembly's detailed herein and/or variations thereof that are utilized to advance and/or retract the electrode array are configured to be utilized with an insertion tool that is handheld instead of being attached to arm 750 system 400. To this end, FIG. 35 depicts an exemplary insertion tool 8200 that includes actuator apparatus 7720 as seen. Hereinafter, the reference will often be made to actuator apparatus 7720 as utilized in conjunction with other components detailed herein. Any disclosure herein of the utilization of actuator apparatus 7720 in conjunction with other teachings detailed herein corresponds to a disclosure of the utilization of the actuator apparatus 8123 or any of the other actuator apparatuses detailed herein or variations thereof utilized to grip and support and/or insert the electrode array into the cochlea. FIG. 35 depicts a connector 67405 in signal communication with an actuator apparatus 7720, which connector is connected to connector 7407, which in turn is connected to a lead which extends to the control unit. In an exemplary embodiment, the surgeon holds the tool 8200 in the traditional manner of use, but the control unit controls the actuation of the actuator 7720 to advance and/or retract the electrode array. In an exemplary embodiment, the surgeon or other healthcare professional can exercise override control over the insertion of the electrode array and/or the retraction of the electrode array. For example, switching components of the like or other types of input devices can be located on the tool 8200 so that the surgeon or the like can provide input into the system of which the tool 8200 is a part. In an alternate embodiment, the tool 8200 can include an input device that interacts with the surgeon, where the surgeon provides the direction to the system advance and/or retract the electrode array, but the control unit evaluates the inputs from the surgeon and controls the actuation accordingly. By way of example only and not by way of limitation, such a system can be analogous to a fly by wire system on an aircraft, where the pilot moves the controls in a manner correlated to the direction that the pilot wants the aircraft to move, and the flight control system controls everything else to achieve the desired outcome. Note also that any the other actuators detailed herein and/or variations thereof can be part of a system that is operated in a similar manner. By way of example only and not by way of limitation, the system 400 can be configured such that the surgeon pushes on the arm 7510 to move the insertion guide is desired, but the system 400 moves the arm 7510 using actuators. That is, the system 400 is configured to sense or otherwise detect the force is applied on to the structure thereof by the surgeon, and then determine what actuator action should be executed so as to position the insertion guide at the desired location in a manner analogous to fly by wire.

It is noted that the electrical lead assembly and the connectors thereof depicted in FIG. 35 can be applicable to any of the insertion guides detailed herein and/or variations thereof so as to place the insertion guide in general, and the actuator assembly thereof in particular, into signal communication with the control unit or other controllers of the system. Note also that in an exemplary embodiment, the lead apparatus depicted in FIG. 35 can be utilized to also convey the other signals detailed herein and/or variations thereof with respect to the other functionalities associated with the insertion guides. Alternatively, and/or in addition to this, the other lead apparatuses detailed herein and variations thereof can be utilized to convey the signals from the actuator apparatus 7720 to the control unit or the like when the insertion guides detailed above are utilized in conjunction with the actuator assembly so as to provide a machine drive to advance and/or retract the electrode array. Any device, system and/or method of communication between any functional component of any of the insertion guides detailed herein and/or variations thereof with a control unit and/or vice versa and/or the implantable component of the electrode array, etc., can be utilized in at least some exemplary embodiments.

It is also noted that while the embodiments detailed herein have been directed towards an electrode array guide, it is also noted that in some alternate embodiments, an electrode array support is instead utilized, which support may not necessarily guide the electrode array, but otherwise might simply support the electrode array proximate to the cochlea. Note that in an electrode array support can also be an electrode array guide, and vice versa.

In view of the above, it can be understood that in an exemplary embodiment, there is an apparatus, such as any of the insertion guides detailed herein and/or variations thereof, that includes an electrode array support, and an actuator. In at least some of these exemplary embodiments, the apparatus is configured to inserts an electrode array into cochlea by a controlled actuation of the actuator. In an exemplary embodiment of such an exemplary embodiment, the controlled actuation is at least partially based on electrical phenomenon of the recipient. Some additional details of such will now be described.

Embodiments of the actuators and/or the insertion devices/robots herein, such as those shown in the figures just described, and/or the figures below, can be utilized to gather data regarding the spatial features that are utilized in the method systems and/or devices disclosed herein. In an exemplary embodiment, the detailed rollers, or more specifically, the movement of the rollers, can be utilized to gauge the distance of insertion of the electrode array. The idea being is that the outer circumference of the rollers will be known, and thus the angular rotation of the rollers can be correlated to movements of the array if there is no slippage between the rollers and the array. In this regard, the systems and devices can be configured so that the angular rotation can be determined, and this can be provided to the overall system to deduce the distance that the array has been inserted or withdrawn. But again, the other types of sensors can also be utilized. And note that while some embodiments have been described in terms of a device that has an automated insertion/retraction system, in other embodiments, the insertion devices do not have such, and instead are simply utilizes a guide or the like, where the surgeon's hand is utilized to apply the insertion and/or removal force. Any of the robotic devices disclosed herein can correspond to the robotic devices or otherwise the actuators described above with respect to the various embodiments. In some embodiments, the surgeon or other healthcare professional utilizes some of the robotic devices, while in other embodiments, the overall system controls these robotic devices.

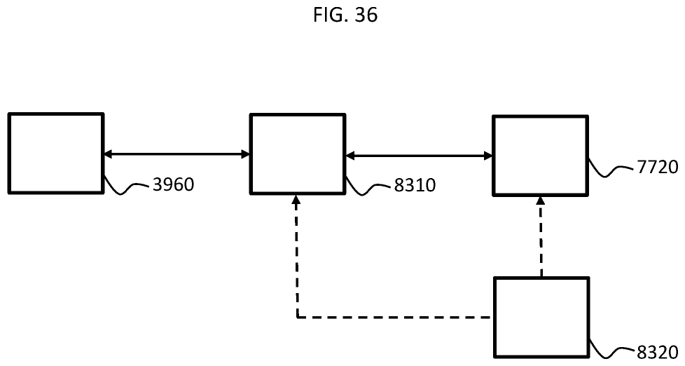

FIG. 36 depicts an exemplary functional schematic of an exemplary system that includes the test unit 3960 detailed above in signal communication with a control unit 8310 which is in turn in signal communication with the actuator assembly 7720. The test unit and the control unit can be one and the same in some embodiments.

It is also noted that in some embodiments, there is no control unit and/or there is no actuator assembly. That is, the system can be a purely test system, which conveys information to the surgeon or other healthcare professional to instruct (e.g., the output of the control unit and/or the test unit can be instead an instruction as opposed to a control signal) or otherwise provide an indication of the phenomenon to the surgeon or other healthcare professional.

Also functionally depicted in FIG. 36 is the optional embodiment where an input device 8320 is included in the system (e.g., which could be on an embodiment where the actuator assembly 7720 is part of a hand tool or where actuator assembly 7720 is part of an insertion guide, where the input device 8320 is located remote from the insertion guide, which could be part of a remote unit 440). In an exemplary embodiment, the input device 8320 could be the trigger for 54 and/or 464 of the remote control unit 440. In an exemplary embodiment, the input device 8320 could be a trigger on the tool 8200. Again, in an exemplary embodiment, the input device 8320 can be utilized to enable advancement and/or withdrawal of the electrode array, and the system 400 could control the advancement and/or withdrawal based on an automated protocol or some other flyby wire type system. In the embodiment of FIG. 36, the input device 8320 can be in signal communication directly to the actuator assembly 7720, and/or in signal communication with the control unit 8310.

In an exemplary embodiment, control unit 8310 can correspond to the remote unit 440. That said, in an alternate embodiment, remote unit 440 can be a device that is in signal communication with control unit 8310. Indeed, in an exemplary embodiment, input device 8320 can correspond to remote control unit 440.

More particularly, control unit 8310 can be a signal processor or the like or a personal computer or the like or a mainframe computer or the like etc., that is configured to receive signals from the test unit 3960 and analyze those signals to evaluate an insertion status of the electrode array. More particularly, the control unit 8310 can be configured with software the like to analyze the signals from test unit 3960 in real time and/or in near real time as the electrode array is being advanced into the cochlea by actuator assembly 7720. The control unit 8310 analyzes the input from test unit 3960 as the electrode array advanced by the actuator assembly 7720 and evaluates the input to determine if there exists an undesirable insertion status of the electrode array and/or evaluates the input to determine if the input indicates that a scenario could occur or otherwise there exists data in the input that indicates that a scenario is more likely to occur relative to other instances where the insertion status of the electrode array will become undesirable if the electrode array is continued to be advanced into the cochlea, all other things remaining the same (e.g., insertion angle/trajectory, etc., which can be automatically changed as well via—more on this below). In an exemplary embodiment, upon such a determination, control unit 8310 could halt the advancement of the array into the cochlea by stopping the actuator(s) of actuator assembly 7720 and/or could slow the actuator(s) so as to slow rate of advancement of the electrode array into the cochlea and/or could reverse the actuator(s) so as to reverse or otherwise retract the electrode array within the cochlea (either partially or fully). In at least some exemplary embodiments, control unit 8310 can be configured to override the input from input unit 8320 input by the surgeon or the user or the like of the systems herein.

In an exemplary embodiment, the outputs of test unit 3960 corresponds to the outputs indicated herein. Alternatively, and/or in addition to this, input into control unit 8310 can flow from other sources. Any input relating to the measurement of voltage associated executing the teachings herein into control unit 8310 can be utilized in at least some exemplary embodiments.

In an exemplary embodiment, control unit 8310 can be configured to determine, based on the input from test unit 3960, whether the electrode array has come into contact with the basilar membrane of the cochlea and/or that one or more of the anomalous electrode positions has occurred and/or whether there exists an increased likelihood that such will occur, and automatically control the actuator assembly 7720 accordingly. In an exemplary embodiment, control unit 8310 does not necessarily determine that such an insertion status exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” the actual insertion status or the forecasted insertion status of the electrode array, but instead need only be able to control the actuator assembly 7720 based on the input.

In an exemplary embodiment, control unit 8310 can be configured to determine, based on the input from test unit 3960, the insertion depth of the electrode array and/or a forecasted insertion depth of the electrode array, and automatically control the actuator assembly 7720 accordingly. In an exemplary embodiment, control unit 8310 does not necessarily determine the insertion depth or forecasted insertion depth, but instead is programmed or otherwise configured so as to control the actuator assembly 7720 according to a predetermined regime based on the input from the test unit 3960. That is, the control unit 8310 need not necessarily “understand” otherwise “know” the actual insertion depth or the forecasted insertion depth of the electrode array, but instead need only be able to control the actuator assembly 7720 based on the input.

In an exemplary embodiment, control unit 8310 can be configured to determine, based on the input from test unit 3960, executing, for example, the methods/techniques disclosed herein, whether the electrode array has buckled and/or bent and/or any other anomalous electrode location as disclosed herein or otherwise may be the case and/or whether there exists an increased likelihood that such will occur, and automatically control the actuator assembly 7720 accordingly. In an exemplary embodiment, control unit 8310 does not necessarily determine that such buckling and/or bending exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” that the electrode array has actually buckled or will buckle in the future, but instead need only be able to control the actuator assembly 7720 based on the input.

Thus, it can be understood that there is an apparatus that is configured to receive input indicative of the electrical phenomenon/phenomena inside the recipient, and develop data indicative of a position of the electrode array within the cochlea based on the input. (It is briefly noted that unless otherwise specified, the singular term phenomenon also includes a disclosure of the plural thereof, and vis-a-versa, as is also the case with the disclosure of data). Still further, such an exemplary embodiment can be configured to adjust the control of the actuation of the actuator based on the developed data indicative of the position of the electrode array.

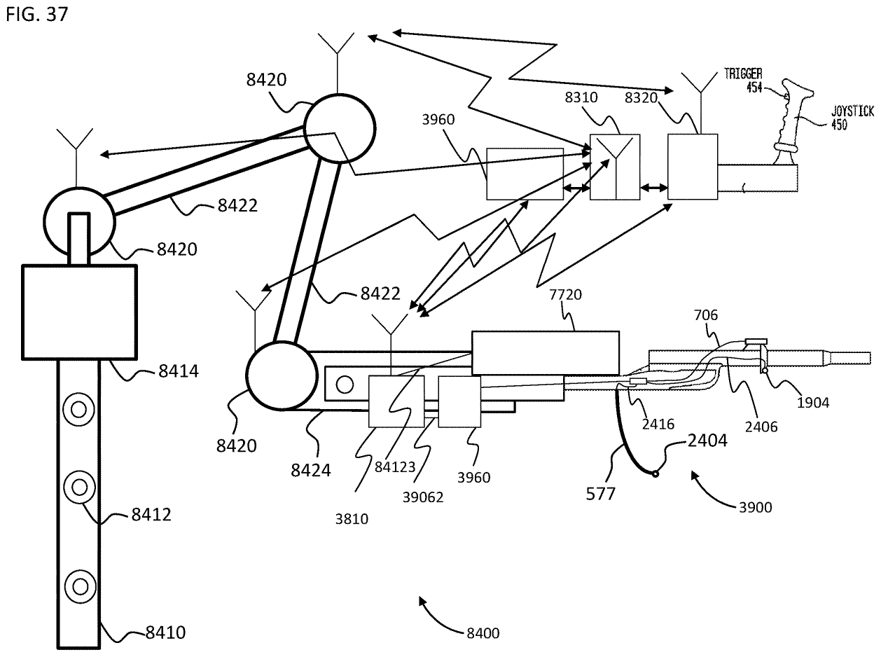

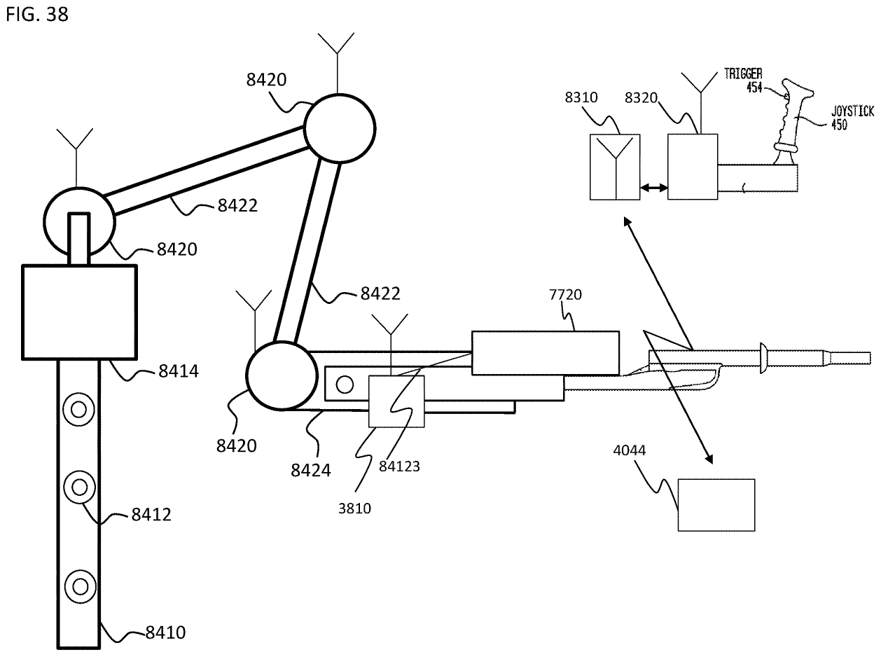

To be clear, while the embodiment detailed above is focused on controlling the actuator assembly 7720 based on data from the system so as to control the advancement and/or retraction of the electrode array based on the data disclosed herein and, in an alternate embodiment, the system 400 controls one or more other actuators of the robot apparatus of system 400. These one or more other actuators can be exclusive from the actuator assembly 7720, or can include the actuator assembly 7720. In this regard, FIG. 37 depicts an exemplary robot apparatus 8400, that includes the insertion guide 3900 detailed above with respect to the integration of a system ad disclosed herein therewith mounted on arm 8424 utilizing bolts in a manner concomitant with that detailed above. In an exemplary embodiment, robot apparatus 8400 has the functionality or otherwise corresponds to that of the embodiment of FIG. 29. In this regard, any functionality associated or otherwise described with respect to the embodiment of FIG. 29 corresponds to that of the embodiment of FIG. 37, and vice versa. In this exemplary embodiment, the actuator apparatus 7720 is in signal communication with unit 3810 via electrical lead 84123. In this regard, signals to and/or from the actuator assembly 7720 can be transmitted to/from the antenna of unit 8310 (in FIG. 38, the “Y” shaped elements are antennas) and thus communicated via lead 84123. It is briefly noted that while the embodiment depicted in FIG. 37 utilizes radiofrequency communication, in alternate embodiments, the communications can be wired. In an exemplary embodiment both can be utilized.

The robot apparatus 8400 includes a recipient interface 8410 which entails an arch or halo like structure made out of metal or the like that extends about the recipient's cranium or other parts of the body. The interface 8410 is bolted to the recipient's head via bolts 8412. That said, in alternate embodiments, other regimes of attachment can be utilized, such as by way of example only and not by way of limitation, strapping the robot to the recipient's head. In this regard, the body and interface 8410 can be a flexible strapping can be tightened about the recipient's head.

Housing 8414 is located on top of the interface 8410, as can be seen. In an exemplary embodiment, housing 8414 includes a battery or the like or otherwise provides an interface to a commercial/utility power supply so as to power the robot apparatus. Still further, in an exemplary embodiment, housing 8414 can include hydraulic components/connectors to the extent that the actuators herein utilize hydraulics as opposed to and/or in addition to electrical motors. Mounted on housing 8414 is the first actuator 8420, to which arm 8422 is connected in an exemplary embodiment, actuator 8420 enables the components “downstream” (i.e., the arm connected to the actuator, and the other components to the insertion guide) to articulate in one, two, three, four, five or six degrees of freedom. A second actuator 8420 is attached to the opposite end of the arm 8422, to which is attached a second arm 8422, to which is attached a third actuator 8420, to which is attached to the insertion guide attachment structure 8424. Elements 8422 and 8424 can be metal beams, such as I beams or C beams or box beams, etc. actuators 8420 can be electrical actuators and/or hydraulic actuators.

As can be seen, each actuator 8420 is provided with an antenna, which antenna is in signal communication with the control unit 8310. In an exemplary embodiment, control unit 8310 can control the actuation of those actuators 8420 so as to position the insertion guide 3900 at the desired position relative to the recipient. That said, in an alternate embodiment, a single antenna can be utilized, such as one mounted on housing 8414, which in turn is connected to a decoding device that outputs a control signal, such as a driver signal based on the decoded RF signal, to the actuators 8420 (as opposed to each actuator having such a device), which control signals can be provided via a wired system/electrical leads extending from housing 8414 to the actuators. Note also that in some alternate embodiments, control unit 8310 is in wired communication with the actuators, either directly or indirectly, and/or is in wired communication with the decoding device located in the housing 8414. Any arrangement that can enable control of the robot apparatus in general, and the actuators thereof in particular, via control unit 8310 can be utilized in at least some exemplary embodiments.

Note also that while the embodiment depicted in FIG. 37 is such that the actuators 8420 must actuate so as to extend the intracochlear portion of the insertion guide into the cochlea, in an alternate embodiment, as noted above, the insertion guide can be mounted on a rail system or the like, wherein a cylindrical actuator or the like pushes the insertion guide in a linear manner into the cochlea and withdrawals the insertion guide in the linear manner from the cochlea. In an exemplary embodiment, this actuator apparatus can enable one degree of freedom movements of the insertion guide, while in other embodiments, this actuator apparatus can enable two or three or four or five or six degrees of freedom. Indeed, in an exemplary embodiment, this actuator apparatus can enable movement only in a linear direction, but can enable rotation of the insertion guide about the longitudinal axis thereof. Any arrangement of actuator assemblies that will enable the insertion guide to be positioned relative to the cochlea and/or inserted into the cochlea via robotic positioning thereof can be utilized in at least some exemplary embodiments.

Any control unit and/or test unit or the like disclosed herein can be a personal computer programs was to execute one or more or all of the functionalities associated there with are the other functionalities disclosed herein. In an exemplary embodiment, any control unit and/or test unit or the like can be a dedicated circuit assembly configured so as to execute one or more or all of the functionalities associated there with or the other functionalities disclosed therein. In an exemplary embodiment, and the control unit and/or test unit or the like disclosed herein can be a processor or the like or otherwise can be a programmed processor.

FIG. 38 depicts another exemplary embodiment, as seen. FIG. 38 presents such an exemplary embodiment, with the links between the antennas removed for clarity. Testing system 4044 detailed shown in signal communication with control unit 8310. In this exemplary embodiment, system 4044 corresponds to that detailed above vis-à-vis determining anomalous electrode location with the exception that it is entirely divorced from the insertion guide, save for the communication between system 4044 and the control unit 8310, to the extent such is relevant for the purposes of discussion, where control unit 8310 is in signal communication with one or more of the assemblies of the robot apparatus, such as the actuator assembly 7720. Here, during insertion, and/or prior to insertion and/or after insertion, the system 4044 monitors or otherwise measures electrical phenomenon detailed herein and communicates those measurements and/or the analysis thereof to control unit 8310, which analyzes those signals and develops a control regime for electrode array insertion and/or electrode array positioning based on those signals. Note also that in some exemplary embodiments, the system 4044 can have multiple measurement electrodes and/or signal generators/sources of acoustic signal generation, some of which are part of the robot apparatus, and some of which are separate from the robot apparatus, all of which are part of system 4044. Alternatively, these various components of the system 4044 can communicate with test unit 3960. Such can have utilitarian value with respect to a scenario where measurements are first taken prior to placing the electrode array near the cochlea and after inserting the electrode array into the cochlea, where it is undesirable to have the insertion guide and/or electrode array support proximate the cochlea. Any device, system, and/or method that will enable controlled movement of the electrode array relative to the cochlea based on electrical phenomenon associated with the recipient/based on electrical characteristics associated with the recipient can be utilized in at least some exemplary embodiments.

Again, the test unit and the system 4044 can be one and the same in some embodiments, and in some embodiments, functionality can be bifurcated between the two as separate units. Indeed, 4044 in FIG. 38 can be a proxy for the control unit and/or the test units detailed above.

In view of the above, it can be seen that some embodiments provide for the automatic detection of a fold over array, a dislocation, bowing or buckling, or other phenomenon, in patients with cochlear implants in an objective manner, and such can provide an automated method for identifying the affected area. Again, the teachings herein can be executed without or in addition to medical imaging tests (e.g., CT scan, X-ray, etc.), or otherwise requiring the recipient/patient to be exposed to radiation during the process of obtaining medical images, and/or subsequent analysis by an expert to assess the correct insertion of the electrode holder and/or measuring neuronal activation after stimulation. In some embodiments, the teachings herein can be executed with methods to attempt to detect neural activation, and can still provide the above reliability in a scenario where there is no neuronal response due to several causes not related to the orientation of the array.

Any method action and/or functionality disclosed herein where the art enables such corresponds to a disclosure of a code from a machine learning algorithm and/or a code of a machine learning algorithm and/or a product of machine learning for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. In an embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code—code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

And to be clear, in an exemplary embodiment, there are products of machine learning algorithms (e.g., the code from the trained machine learning algorithm) that are included in any one or more of the systems/subsystems detailed herein, that can be utilized to analyze any of the data obtained or otherwise available disclosed above that can be utilized or otherwise is utilized to evaluate the data obtained herein. This can be embodied in software code and/or in computer chip(s) that are included in the system(s).

An exemplary system includes an exemplary device/devices that can enable the teachings detailed herein, which in at least some embodiments can utilize automation. That is, an exemplary embodiment includes executing one or more or all of the methods and/or functionalities detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

Prediction can be represented by an algorithm where circuitry receives the input (embodied in an analogue or a digital signal), where the input suite converts the “physical” input into electronic signals using analog to digital converters for example, or in the case of the input suite corresponding to an Internet server, receives the digital signal from a remote location, and the digital data is stored in a memory and/or received by the electronics. The electronics, which is a result of the machine learning, takes the digital signal and deconstructs the digital signal to evaluate properties, and then, using its “knowledge” from its training, provides an output corresponding to the prediction. By analogy, the operation is analogous to how a human being “predicts” how he or she will function if he or she foregoes a meal for example, or stays up all night, or if he or she drinks 5 cups of coffee in one hour. Past experience informs the future results, the prediction.

The cohort comparator can be a database such as Microsoft™ Access, where the computer automatically matches the data instead of the human matching the data. The results of machine learning and/or a product thereof can be used to perform the automatic matching.

In an exemplary embodiment, the cohort comparator is a computer chip and/or a computer circuit. The cohort comparator can be electronics. In an exemplary embodiment, cohort comparison can be represented by an algorithm where circuitry receives the input (embodied in an analogue or a digital signal), where the input suite converts the “physical” input into electronic signals using analog to digital converters for example, or in the case of the input suite corresponding to an Internet server, receives the digital signal from a remote location, and the digital data is stored in a memory and/or received by the electronics. The electronics takes the digital data and “looks” for certain strings of zeros and ones that correspond to a match with signatures/identifiers linked to prestored data regarding performance capabilities. The data linked to the signatures/identifiers is the cohort identified.

Embodiments can include a system and/or simply an embodiment that includes a non-transitory computer readable medium having recorded thereon, a computer program for executing at least a portion of a method, the computer program including code for executing any one or more of the method actions and/or functionalities detailed herein. Thus, any disclosure herein of a method action or functionality corresponds to a disclosure of a non-transitory computer readable medium having programed thereon code to execute one or more of those actions and also a product to execute one or more of those actions.

Embodiments include any functionality disclosed herein and/or method action disclosed herein being executed by a computer chip, a processor, software, logic circuitry and/or electronics, and all are not mutually exclusive. Any circuit that can enable the teachings herein can be used providing that the art enables such. Thus, in the interests of textual economy, and disclosure herein of a functionality of an article of manufacture corresponds to any one or more of the aforementioned structures being configured to execute such and otherwise for such, and the same is for any method action disclosed herein, where any such action corresponds to a disclosure of any one or more of the aforementioned structures being configured to execute such and otherwise for such.

Any disclosure herein of a processor corresponds to a disclosure in an embodiment of a non-processor device or a combined processor-non-processor device where the non-processor is a result of machine learning. Embodiments can include a link from the cloud to a clinic to pass information back and forth, enabling the remote processing noted above and/or enabling the obtaining of additional data for retraining purposes. Information can be uploaded to the cloud to the clinic, where the information can be analyzed. Another exemplary system includes a smart device, such as a smart phone or tablet, etc., that is running a purpose built application to implement some of the teachings detailed herein. This can be used by the clinician, and can contain at least the front end portions of the systems and devices detailed herein, or otherwise provide the interface portal to the back end. Any disclosure herein of a processor corresponds to a disclosure of a non-processing device, or includes non-processing devices, such as a chip or the like that is a result of a machine learning algorithm or machine learning system, etc.

It is further noted that any disclosure of a device and/or system detailed herein also corresponds to a disclosure of otherwise providing that device and/or system and/or utilizing that device and/or system.

It is also noted that any disclosure herein of any process of manufacturing or providing a device corresponds to a disclosure of a device and/or system that results therefrom. Is also noted that any disclosure herein of any device and/or system corresponds to a disclosure of a method of producing or otherwise providing or otherwise making such. Any functionality of a device disclosed herein corresponds to a disclosure of a method action corresponding to that functionality. Any method action disclosed herein corresponds to a disclosure of a device and/or system for executing such, providing that the art enables such.

An exemplary system includes an exemplary device/devices that can enable the teachings detailed herein, which in at least some embodiments can utilize automation, as will now be described in the context of an automated system. That is, an exemplary embodiment includes executing one or more or all of the methods detailed herein and variations thereof, at least in part, in an automated or semiautomated manner using any of the teachings herein.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so in an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A method, comprising:
advancing, as part of an implantation procedure into a human, at least a first portion of an electrode array into a cochlea of the human during a first temporal period;
providing information to a computer system, the provided information including spatial based data relating to the electrode array during the implantation procedure of the electrode array into the human;
receiving information based on an evaluation by the computer system of the provided information, the evaluation having taken into account a history of the spatial based data provided in the provided information, the received information being a recommendation as to how to move the electrode array during second temporal period following the first temporal period; and
moving the electrode array according to the received information.

2. The method of claim 1, wherein:
the history includes the most recent movement being the insertion of the array further into the cochlea and the electrode array is almost fully inserted;
the history includes that the array was stationary, and a pose of the array was that the electrode array is clinically against a modiolus wall of the cochlea; and
the received information is to move the electrode array further into the cochlea.

3. The method of claim 1, wherein:
the history includes the most recent movement being the insertion of the array further into the cochlea and the electrode array is at least almost fully inserted;
the evaluation has taken into account current spatial based data of the array;
a current pose is that a mid-section of the electrode array is clinically away from a modiolus wall of the cochlea; and
the received information is to move the electrode array backwards.

4. The method of claim 3, further comprising:
receiving a second information after the received information, the second information being to advance the electrode array into the cochlea and then to move the electrode array backwards;
advancing the array and then moving the electrode array backwards based on the second information;
receiving a third information to leave the electrode array at the location where it was moved backwards; and
leaving the electrode array at the location where it was moved backwards based on the second information.

5. The method of claim 1, wherein:
the history includes the most recent movement being to insert the array further into the cochlea and the electrode array is less than ⅔rds of the way fully inserted;
the current pose is that an apical section of the electrode array is clinically away from a modiolus wall of the cochlea; and
the received information is to move the electrode array backwards by a relatively significant amount, and then move the electrode array forward by more than an amount corresponding to the relatively significant amount.

6. The method of claim 5, further comprising:
receiving a second information after the received information, the second information being to advance the electrode array into the cochlea further,
advancing the array further based on the second information;
receiving a third information after the received second information, the third information being to advance the electrode array into the cochlea further.

7-8. (canceled)

9. The method of claim 1, further comprising:
providing demographic and/or physiological information about the human to the computer system, wherein the evaluation has also taken into account the provided demographic and/or physiological information.

10-11. (canceled)

12. A non-transitory computer readable medium having recorded thereon, a computer program for executing at least a portion of a method, the computer program including:
code for receiving input based on at least one of past movement, past pose, past status, current movement, current pose or current status of an electrode array at least partially in a human during an implantation procedure of the electrode array in the human; and
code for automatically analyzing data based on at least a portion of the input to develop a recommend action to be taken in the implantation procedure, wherein the analyzed data includes:
a current position of the electrode array; and
at least one of:
at least one movement of the array during a first temporal period prior to a current temporal period;
at least one past pose of the array during the first temporal period; or
at least one past status of the electrode array during the first temporal period.

13. The medium of claim 12, wherein:
the first temporal period extends a period of at least 10 seconds;
the analyzed data includes at least two discretized movements of the electrode array.

14. The medium of claim 13, wherein:
the input is based on at least one of past movement or current movement, and at least one of the movements of the at least one of the past movement or current movement is continuous for at least 5 seconds; and
the medium further includes discretizing the continuous movement into the two discretized movements.

15. The medium of claim 12, wherein:
the analyzed data includes at least one past pose of the array during the first temporal period; and
the current position is based on at least a current pose of the array.

16. The medium of claim 12, wherein:
the analyzed data includes the at least one movement of the array during the first temporal period; and
the analyzed data includes a current movement of the electrode array.

17. The medium of claim 12, wherein:
the analyzed data includes the at least one past status of the electrode array during the first temporal period; and
the analyzed data includes a current status of the electrode array.

18. The medium of claim 12, wherein:
the analyzed data includes at least three discretized movements based on movement(s) during the first temporal period;
the analyzed data includes at least two past poses of the array during the first temporal period; and
the analyzed data includes at least two past statuses of the array during the first temporal period.

19-44. (canceled)

45. A method, comprising:
obtaining (i) medical device positional data and (ii) medical device movement data for a statistically significant number of medical device implantation processes on a human for the medical device; and
analyzing the obtained data to develop a predictive algorithm for a current medical device movement behavior based on the results of the analysis, wherein the predictive algorithm predicts future movement of the current medical device based on input specific to the current medical device, the current medical device not being one of the medical devices of the medical device implantation processes.

46. The method of claim 45, wherein:
the action of analyzing the obtained data is executed using machine learning.

47. The method of claim 46, wherein:
the use of machine learning includes calculating coefficients, parameters, thresholds and/or weights of models by minimizing a cost function.

48. The method of claim 45, wherein:
the action of analyzing the obtained data machine-trains a system that results in the developed predictive algorithm.

49. The method of claim 45, further comprising:
developing a cohort model based on the obtained data, wherein
the action of analyzing the obtained data results in the development of a cohort model based on the obtained data, and
the predictive algorithm uses the cohort model in a brute force manner to predict future movement of the current medical device based on input specific to the current medical device.

50-51. (canceled)

52. The method of claim 45, further comprising:
curation of the obtained data to remove data having records that are sparely populated and/or are low quality.

53-55. (canceled)
